(19) United States
(12) Patent Application Publication
(10) Pub. No.: US 2015/0018740 A1
(43) Pub. Date: Jan. 15, 2015

(54) HEEL FLOAT THERAPEUTIC FOOTWEAR WITH RIGID HEEL CUP

(71) Applicant: OSBORN MEDICAL CORP., Centennial, CO (US)

(72) Inventors: William Donahoe Davis, Denver, CO (US); Ian Patrick MacDonald, Rochester, MN (US); Keith Coulson Ware, Burnsville, MN (US)

(73) Assignee: OSBORN MEDICAL CORP., Centennial, CO (US)

(21) Appl. No.: 14/267,304

(22) Filed: May 1, 2014

Related U.S. Application Data
(60) Provisional application No. 61/882,423, filed on Sep. 25, 2013.
(60) Provisional application No. 61/818,270, filed on May 1, 2013.

(51) Int. Cl. A61F 5/01

(52) U.S. Cl. CPC A61F 5/0127
USPC 602/28; 29/428

(57) ABSTRACT

A heel float therapeutic footwear apparatus for people that spend a large amount of time in bed. The heel float therapeutic footwear apparatus includes a lower leg support portion, a heel float cradle, a foot support portion, a wing support structure, and a heel support structure. The foot support portion extends from the lower leg support portion. The heel support structure is permanently attached to the heel float cradle. A method of making a heel float therapeutic footwear is also disclosed.

### CROSS-REFERENCE TO RELATED APPLICATIONS

This application claims priority to U.S. Provisional Application Ser. No. 61/882,423 filed on Sep. 25, 2013, entitled HEEL FLOAT THERAPEUTIC FOOTWEAR AND RIGID HEEL CUP; and to U.S. Provisional Application Ser. No. 61/818,270 filed on May 1, 2013, entitled HEEL FLOAT THERAPEUTIC FOOTWEAR AND RIGID HEEL CUP; the disclosures of which are incorporated by reference herein in their entireties.

### BACKGROUND

Hospital patients are sometimes bed ridden for long periods of time. In some situations the patient can experience problems associated from lying down in one position for so long. For example, patients with vascular disease need to have good blood circulation to prevent clotting. The proper pre and post-operative footwear is needed to prevent vasoconstriction and promote vasodilation. In addition to maintaining proper blood circulation, the foot must be prevented from pointing in the downward direction or leaning to the side, a problem recognized in the industry that can lead to foot drop. Foot drop is a condition that may occur after lying in bed for some time without getting up or walking. Foot drop is the dropping of the forefoot due to weakness, damage to the peroneal nerve or paralysis of the muscles in the anterior portion of the lower leg. It is characterized by the inability or difficulty in moving the ankle and toes upward and thereby leading to the improper rotation of the foot. Heel or foot ulcers are another condition that can develop as a result of the heel rubbing against the pre or post-operative footwear or pressure being placed on the heel while lying in a hospital bed.

### SUMMARY

In general terms, this disclosure is directed to a heel float therapeutic footwear apparatus. In one possible configuration, and by non-limiting example, a heel float therapeutic footwear apparatus includes a lower leg support portion, a foot support portion, a heel float cradle, and a heel support structure.

One aspect is a heel float therapeutic footwear apparatus including: a lower leg support portion; a foot support portion extending from the lower leg support portion; a heel float cradle having a raised portion, the raised portion configured to elevate a heel of a patient when the patient is lying on the patient's back to reduce pressure on the patient's heel, the heel float cradle defining a recess; and a portion of a heel support structure adapted to be received within the recess of the heel float cradle for protecting the heel and ankle of the patient, the heel support structure forming a suspension bridge thereon to suspend a foot of the patient.

Another aspect is a heel float therapeutic footwear apparatus including: a lower leg support portion configured to support a lower leg of a patient; a foot support portion connected to and extending from the lower leg support portion; a heel float cradle having a raised portion, the raised portion configured to elevate a heel of the patient when the patient is lying on the patient's back to reduce pressure on the patient's heel; and a heel support structure configured to attach to the heel float cradle; the heel support structure being adapted to protect the heel of the patient, the heel support structure including a main body including a posterior wall and a side walls that extend from the posterior wall, the side walls cooperating to define a perimeter of the main body; an interior region being defined by the posterior wall and the side walls; and a tab adapted to be attached to the side walls, wherein an attachment mechanism is used to secure the tab to side walls of the heel float cradle forming a suspension bridge thereon to suspend a foot of the patient.

A further aspect is a method of making a heel float therapeutic footwear apparatus, the method including: forming a lower leg support portion and a foot support portion; forming a heel float cradle within the lower leg support portion; the heel support cradle having a raised portion, the raised portion configured to elevate a heel of the patient when the patient is lying on the patient's back to reduce pressure on the patient's heel; and permanently attaching a heel support structure on the heel support cradle, the heel support structure being adapted to suspend a foot and protect the heel of the patient.

### BRIEF DESCRIPTION OF THE DRAWINGS

FIG. 1 is a perspective view of an example of a heel float therapeutic boot with a wing support structure fastened in accordance with the principles of the present disclosure.

FIG. 2 is a perspective view of the heel float therapeutic boot shown in FIG. 1 with the wing support structure unfastened.

FIG. 3 is a perspective view of the heel float therapeutic boot shown in FIG. 2 with upper overlapping members unfastened.

FIG. 4 is a perspective back view of the heel float therapeutic boot shown in FIG. 1.

FIG. 5 is a component view of a portion of the heel float therapeutic boot shown in FIG. 3 taken along line 5-5.

FIG. 6 is a perspective view of an example of a heel float cradle in an upright position in accordance with the principles of the present disclosure.

FIG. 7 is a cross-sectional view of a portion of the heel float cradle shown in FIG. 6.

FIG. 8 is a perspective view of a portion of the heel float therapeutic boot shown in FIG. 2.

FIG. 9 is a bottom perspective view of the heel float therapeutic boot shown in FIG. 1.

FIG. 10 is a component view of a portion of the heel float therapeutic boot shown in FIG. 2 taken along line 10-10.

FIG. 11 is a perspective view of the heel float cradle shown in FIG. 6 without being in an upright position.

FIG. 12 is an enlarged view of a portion of the heel float cradle shown in FIG. 11.

FIG. 13 is a cross-sectional view of the heel float therapeutic boot shown in FIG. 1 with a lower leg of a patient.

FIG. 14 is a side perspective view of the heel float therapeutic boot shown in FIG. 1.

FIG. 15 is a bottom perspective view of the heel float therapeutic boot shown in FIG. 1 with the wing support structure unfastened.

FIG. 16 is an enlarged view of the wing support structure shown in FIG. 15.

FIG. 17 is a flow chart illustrating a method of making a heel float therapeutic footwear apparatus in accordance with the principles of the present disclosure.

FIG. 18 is a side perspective view of an alternate example of a heel float therapeutic boot with a heel support structure fastened in accordance with the principles of the present disclosure.

FIG. 19 is a cross-sectional view of the heel float therapeutic boot shown in FIG. 18 with a lower leg of a patient.

FIG. 20 is a front perspective view of the heel support structure shown in FIG. 18.

FIG. 21 is a side perspective view of the heel support structure shown in FIG. 18.

FIG. 22 is a back perspective view of the heel support structure shown in FIG. 18.

FIG. 23 is a bottom perspective view of the heel support structure shown in FIG. 18.

FIG. 24 is a flow chart illustrating a method of making a heel float therapeutic footwear apparatus with a heel support structure fastened in accordance with the principles of the present disclosure.

FIG. 25 is a perspective view of a therapeutic boot support including an alternate example of a heel support structure and a heel float cradle in accordance with the principles of the present disclosure.

FIG. 26 is a side perspective view of the therapeutic boot support shown in FIG. 25.

FIG. 27 is a front perspective view of the therapeutic boot support shown in FIG. 25.

FIG. 28 is a left perspective view of the heel float cradle shown in FIG. 25.

FIG. 29 is a side perspective view of the heel float cradle shown in FIG. 28.

FIG. 30 is a back perspective view of the heel float cradle shown in FIG. 28.

FIG. 31 is a left front perspective view of the heel float cradle shown in FIG. 28.

FIG. 32 is a left back perspective view of the heel float cradle shown in FIG. 28 viewed from the bottom.

FIG. 33 is a perspective view of the heel float cradle shown in FIG. 28 viewed from the top.

FIG. 34 is a cross-sectional view taken along line 34-34 of the heel float cradle shown in FIG. 27.

FIG. 35 is a perspective cross-sectional view of the heel float cradle shown in FIG. 34.

FIG. 36 is a perspective view of the heel support structure shown in FIG. 25.

FIG. 37 is a left back perspective view of the heel support structure shown in FIG. 36

FIG. 38 is a back perspective view of the heel support structure shown in FIG. 36.

FIG. 39 is a top perspective view of the heel support structure shown in FIG. 36.

FIG. 40 is a bottom perspective view of the heel support structure shown in FIG. 36.

FIG. 41 is a right perspective view of the heel support structure shown in FIG. 36 viewed from the bottom.

FIG. 42 is a side perspective view of the heel support structure shown in FIG. 36.

FIG. 43 is a cross-sectional plan view taken along line 43-43 of the therapeutic boot support shown in FIG. 26.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

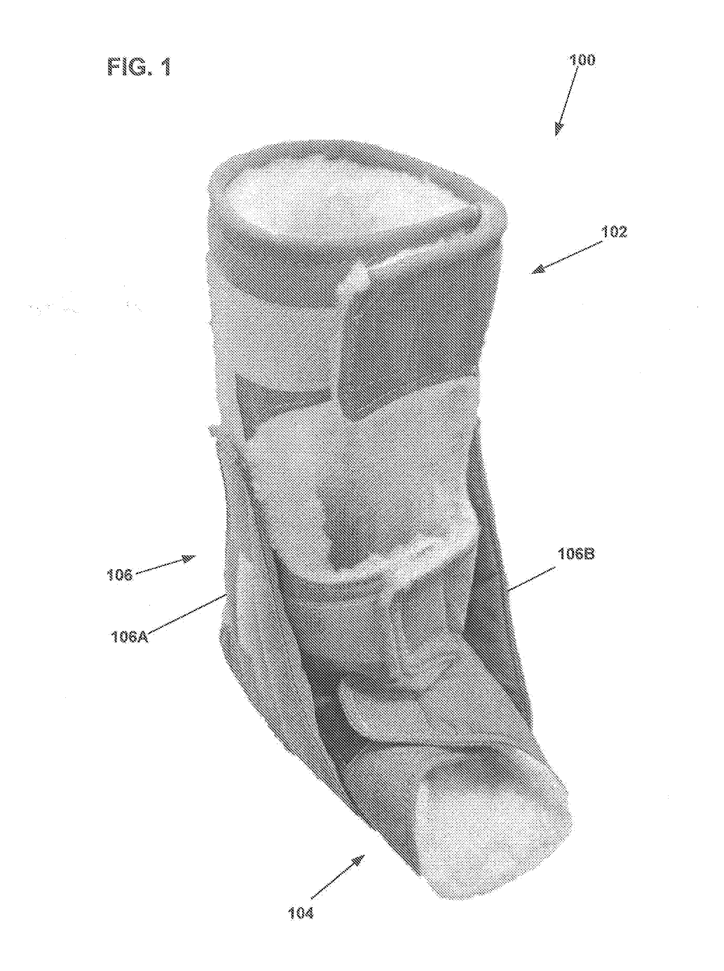

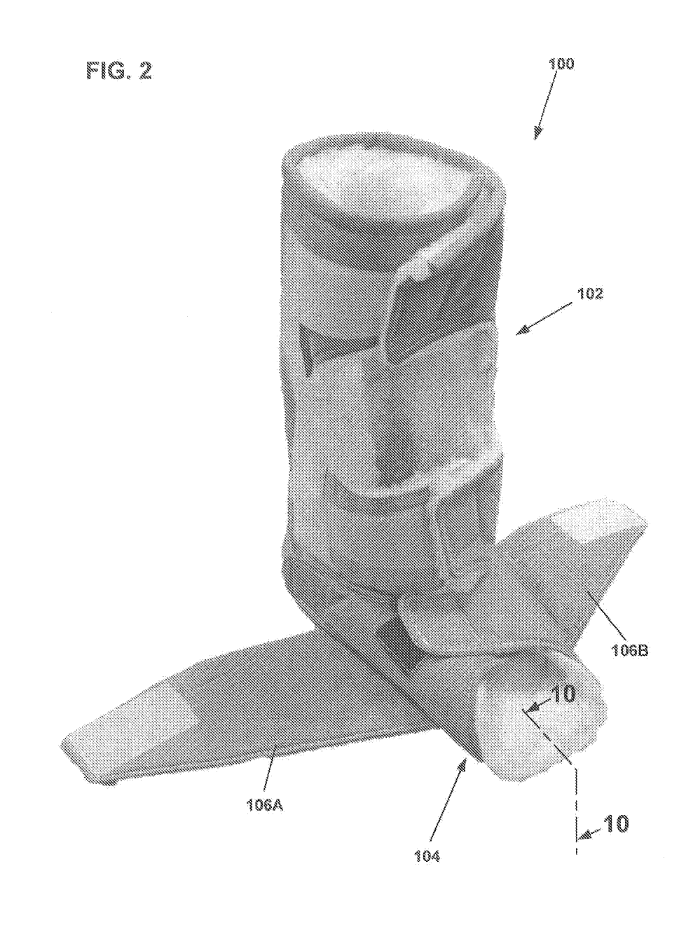

FIGS. 1-2 are perspective views of an example of a heel float therapeutic boot 100. In this example, the heel float therapeutic boot 100 includes a lower leg support portion 102, a foot support portion 104, and a wing support structure 106. In FIG. 1, the wing support structure 106 is configured as being attached to the heel float therapeutic boot 100. In FIG. 2, the wing support structure 106 is shown as being open and unfastened to the heel float therapeutic boot 100.

The heel float therapeutic boot 100 can be worn by hospital patients to help support the foot of a patient and off-load weight from the heel of the patient. The heel float therapeutic boot 100 may help to prevent foot drop by keeping the foot in an upright position thereby preventing the foot from falling over to one side or from pointing toward the foot of the bed while lying down. The heel float therapeutic boot 100 can also be used to elevate the heel so that it does not rub on the bottom of the boot or on the padding. The heel float therapeutic boot 100 supports the foot in a preferred position.

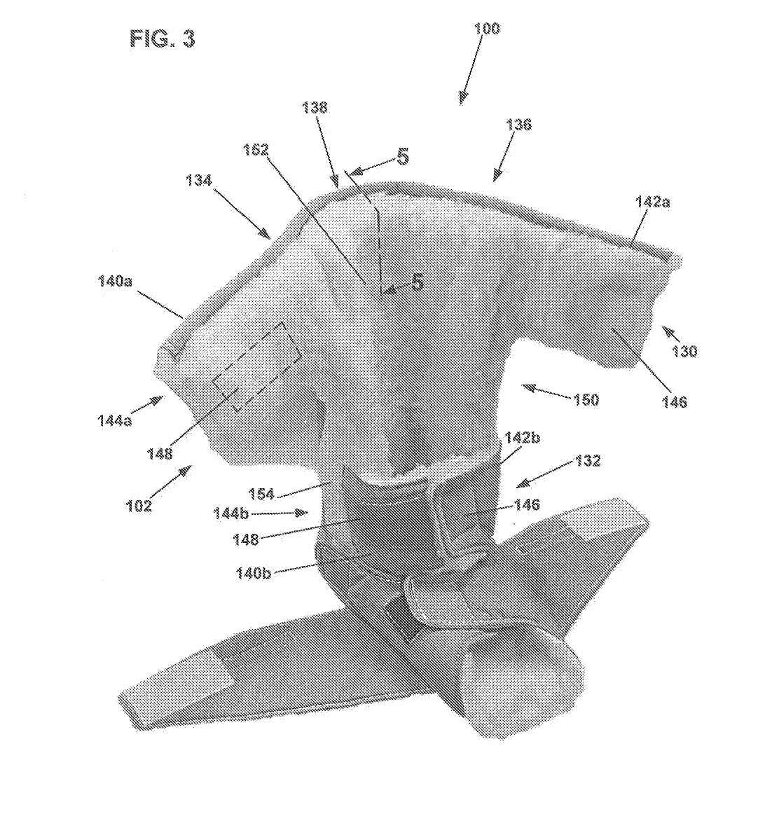

The lower leg support portion 102 may be considered the main body of the heel float therapeutic boot 100 and typically covers the lower leg portion of a patient's leg including at least a portion of the calf and ankle. The lower leg support portion 102 helps protect against pressure ulcers formed on the skin and keeps the legs warm. The lower leg support portion is illustrated and described in more detail with reference to FIGS. 3-7.

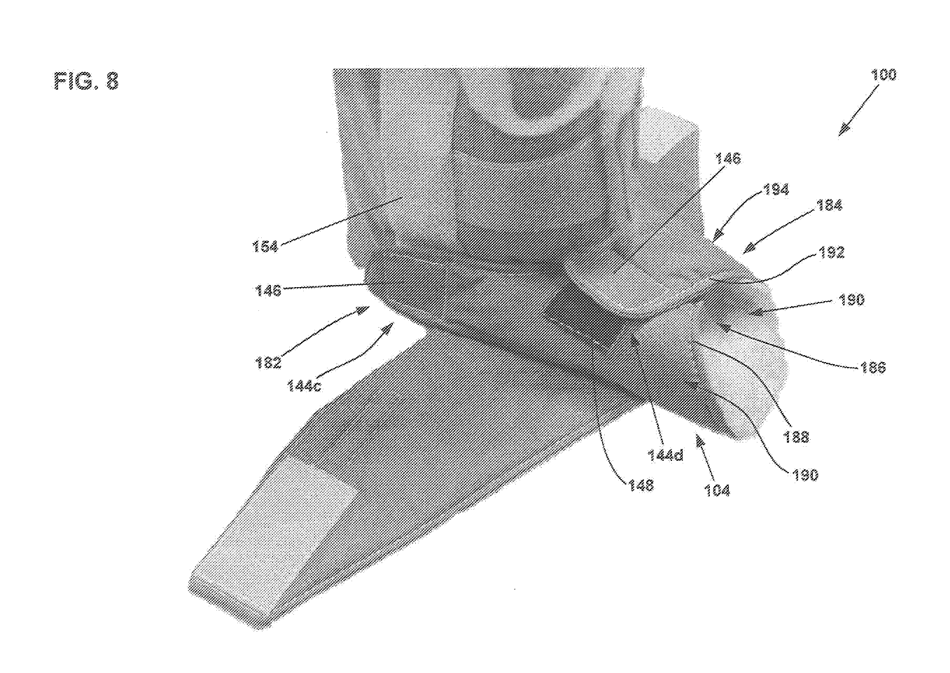

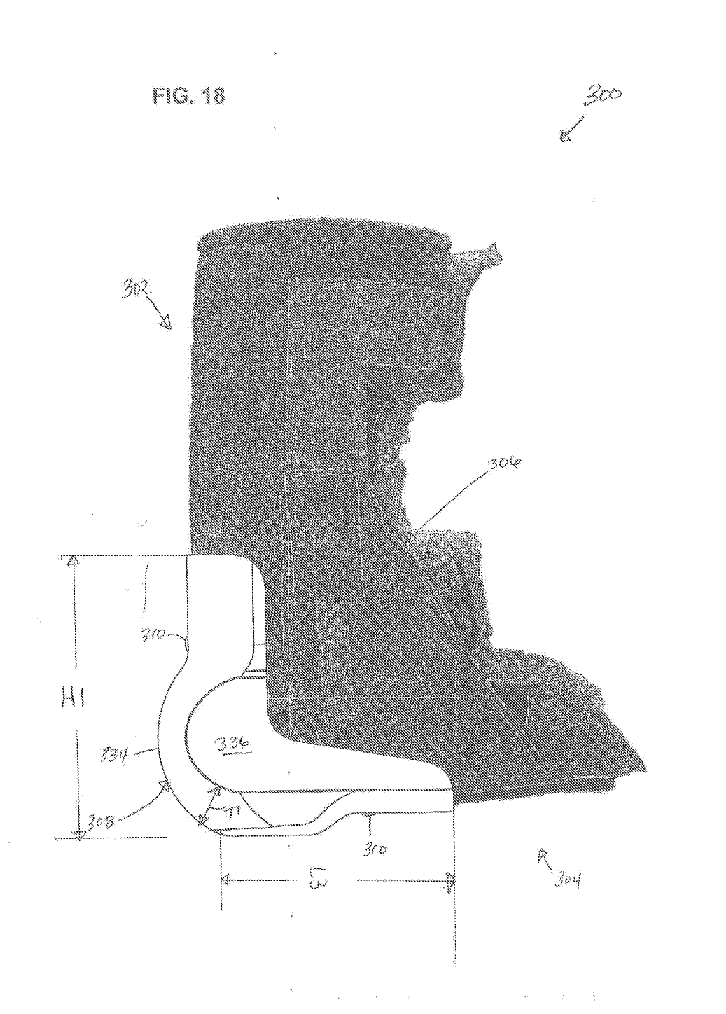

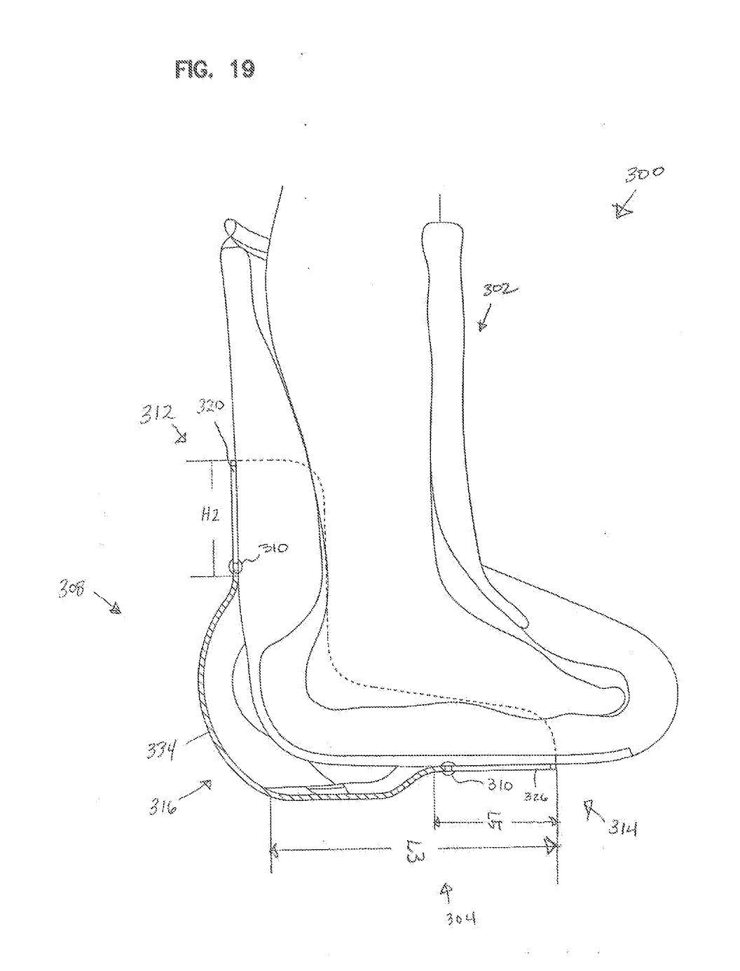

The foot support portion 104 of the heel float therapeutic boot 100 extends adjacent to the lower leg support portion 102 and covers portions of the foot area. The foot support portion 104 helps protect against pressure ulcers formed on the skin and keeps the feet warm. In some embodiments, the foot support portion 104 is connected to the lower leg support portion 102 creating a hinge between the two. In other embodiments, the foot support portion 104 may include a heel support structure 308 (see FIG. 18) fastened to the foot support portion 104 to provide added protection to the heel and ankle and to help prevent pressure ulcers. The foot support portion 104 is illustrated and described in more detail with reference to FIGS. 8-12. The heel support structure 308 is illustrated and described in more detail with reference to FIGS. 18-19.

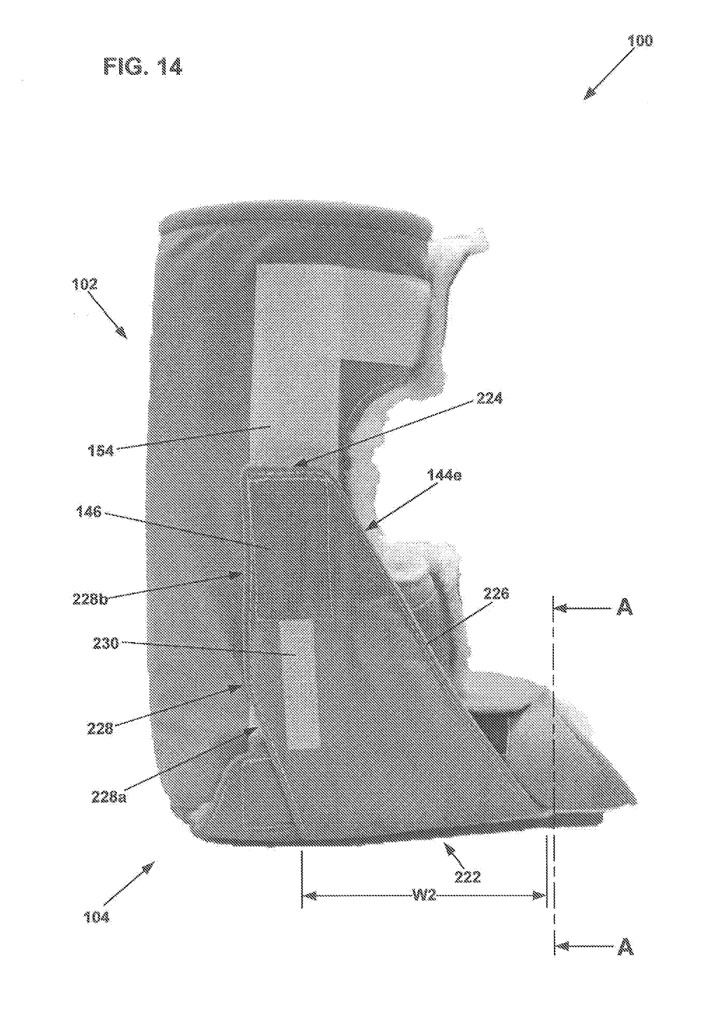

The wing support structure 106 of the heel float therapeutic boot 100 supports the foot in a preferred position. The wing support structure 106 is attached to the heel float therapeutic boot 100 and is configured to be adjustable to obtain the preferred position. The wing support structure 106 is illustrated and described in more detail with reference to FIGS. 14-16.

FIGS. 3-5 illustrate features of the lower leg support portion 102.

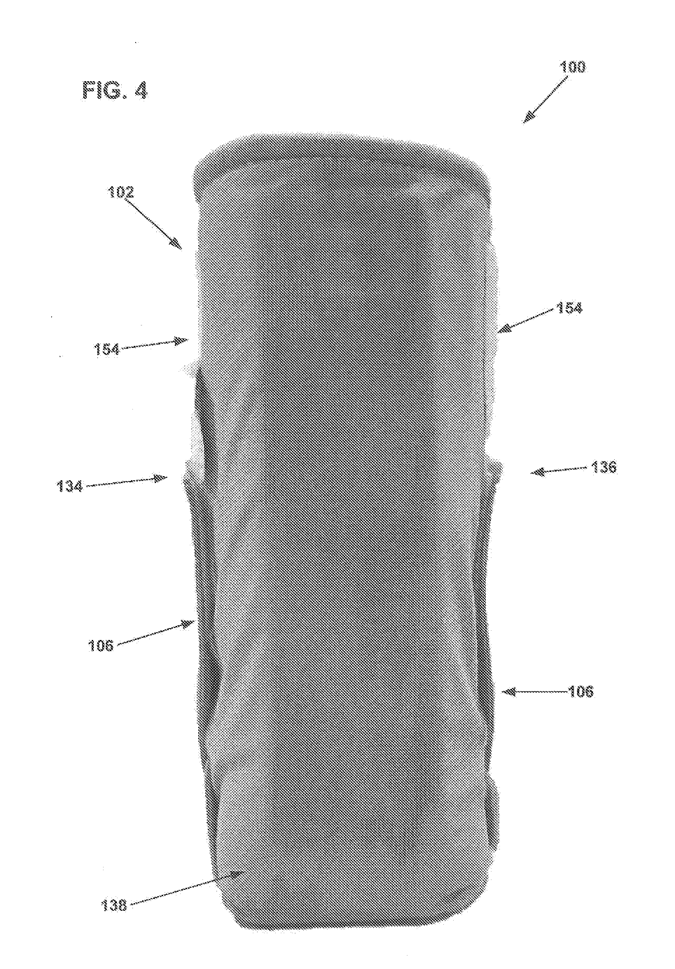

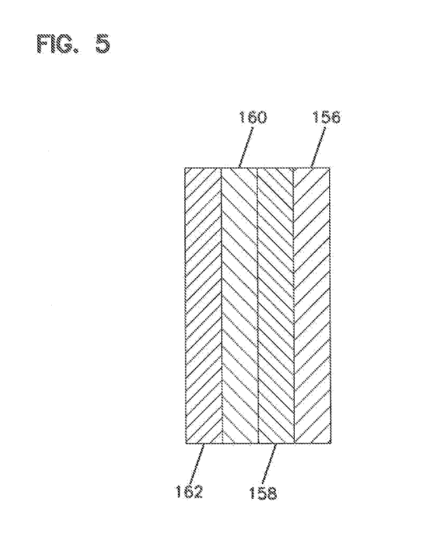

FIGS. 3-4 are perspective front and back views of the lower leg support portion 102. In this example, the lower leg support portion 102 includes an upper overlapping member 130, a lower overlapping member 132, a left side wall 134, a right side wall 136, and a back side wall 138. The lower leg support portion 102 is formed of one piece connecting all of the side walls with both of the overlapping members. The lower leg support portion 102 includes multiple layers that are illustrated and described in detail with reference to FIG. 5.

The upper overlapping member 130 includes a left side panel 140a extending from the left side wall 134 of the lower leg support portion 102 and a right side panel 142a extending from the right side wall 136 of the lower leg support portion 102. The upper overlapping member 130 has a fastening mechanism 144a to connect the left side panel 140a together with the right side panel 142a. In some embodiments, the fastening mechanism 144a includes a hook patch 146 and a loop patch 148. In other embodiments, other fastening mechanisms are used, such as, buttons, adhesive, hooks, clips, clasps, bolts, straps, or combinations of these or other fastening mechanisms. The loop patch 148 is attached to the exterior surface of the left side panel 140a of the upper overlapping member 130 and the hook patch 146 is attached to the right side panel 142a of the upper overlapping member 130. In this example, the hook patch 146 and the loop patch 148 are sewn onto respective panels of the upper overlapping member 130 to be fastened together as the two overlap. In other embodiments, the arrangement and configuration of the hook patch 146 and the loop patch 148 can vary.

The lower overlapping member 132 includes a left side panel 140b extending from the left side wall 134 of the lower leg support portion 102 and a right side panel 142b extending from the right side wall 136 of the lower leg support portion 102. The lower overlapping member 132 has a fastening mechanism 144b to connect the left side panel 140b together with the right side panel 142b. The lower overlapping member 132 may also be provided with a hook patch 146 and a loop patch 148 as the fastening mechanism 144b similar to those described for the upper overlapping member 130. Accordingly, many of the concepts and features for the lower overlapping member 132 are similar to the upper overlapping member 130 previously described herein. In some embodiments, the lower overlapping member 132 is color coded such that the hook patch 146 and loop patch 148 colors are different from other fastening mechanisms, i.e. the upper overlapping member 130. This feature helps to identify for the patient where each overlapping member should be attached if the heel float therapeutic boot 100 becomes crumpled or twisted.

In this example, the upper and lower overlapping members 130, 132 of the lower leg support portion 102 are arranged and configured to wrap and fasten about an anterior portion of the lower leg. In other examples, the lower leg support portion 102 may extend further up the leg. In some embodiments, the upper overlapping member 130 and the lower overlapping member 132 are arranged and configured to define an opening 150 in the front of the lower leg support portion 102 of the heel float therapeutic boot 100. This opening may help provide a cooling feature for the patient when warming is not so critical. It is to be understood that other configurations may be used, such as, but not limited to, a closed configuration having no opening, a slide on boot, or a slip on boot, and combinations of these and other configurations.

Referring to FIG. 4, the left side wall 134 of the lower leg support portion 102 and the right side wall 136 of the lower leg support portion 102 are integrally formed with the back side wall 138 of the lower leg support portion 102 defining a channel 152 (shown in FIG. 3) for receiving a lower leg of a patient. In this example, the left side wall 134 of the lower leg support portion 102 and the right side wall 136 of the lower leg support portion 102 has loop material 154 vertically positioned along the left and right side of the lower leg support portion 102. In this example, the wing support structure 106 is configured to fasten along the loop material 154 positioned on the left and right side of the lower leg support portion 102.

FIG. 5 is a component view of a portion of the heel float therapeutic boot 100 depicting layers of the lower leg support portion 102. In this example, the lower leg support portion 102 includes a fleece material 156, a lining 158, a heel float cradle 160 and exterior fabric 162. The construction and materials used in the heel float therapeutic boot 100 provides the proper support and protection needed for patients.

The fleece material 156 includes the entire lining 158 of the heel float therapeutic boot 100 and is positioned to surround the lower leg of the patient. In this example, the fleece material 156 covers the entire interior of the heel float therapeutic boot 100 to provide for a soft, smooth/comfortable surface to contact the skin without any abrading seams that can cause skin ulcerations. In some embodiments, the fleece material 156 is laminated to the lining 158 of the heel float therapeutic boot 100. In other embodiments, the fleece material 156 can be placed on the lining 158 by other attachment means, such as, adhesive, sewing, or combinations of these or other attachment means.

The lining 158 helps wick moisture away and ventilate the lower leg and foot. Moisture typically develops at regions of sustained contact between the skin and the fleece material 156. In some embodiments, seams can be located between sections of the fleece material 156 and/or lining 158 to minimize contact with the skin. These seams can be sewn or adhesively fixed in the heel float therapeutic boot 100.

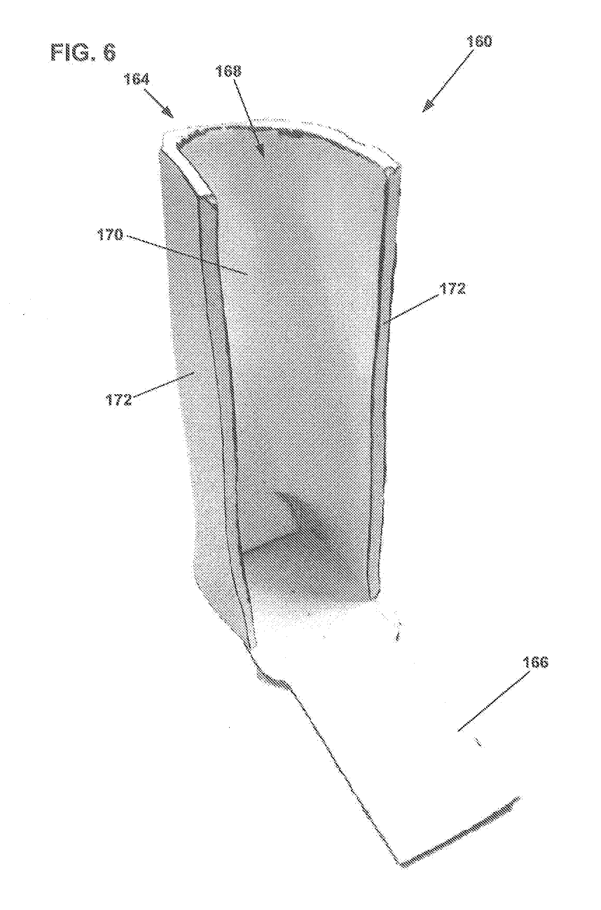

The heel float cradle 160 is typically a relatively dense elastic material that flexes or compresses slightly to provide a resilient interface with the lower leg, for example, foam. In this example, the heel float cradle 160 is positioned between the exterior fabric 162 of the heel float therapeutic boot 100 and the lining 158 of the heel float therapeutic boot 100. In some embodiments, the heel float cradle 160 extends along the length of the lower leg support portion 102 and the foot support portion 104. The heel float cradle 160 is illustrated and described in more detail with reference to FIG. 6.

The exterior fabric 162 is constructed of an air permeable material, for example, a durable velour cloth. In some embodiments, other materials such as a heavy weight cotton fabric, CORDURA® or other fabric or fabric combinations might also be used. The exterior fabric 162 covers the entirety of the heel float therapeutic boot 100.

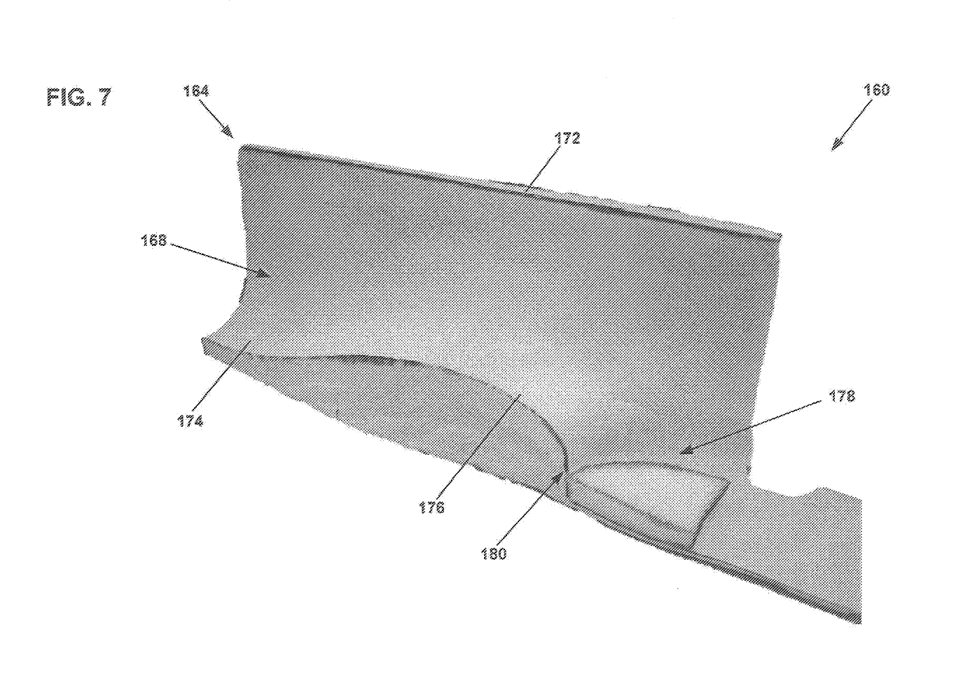

FIGS. 6-7 illustrate features of the heel float cradle 160.

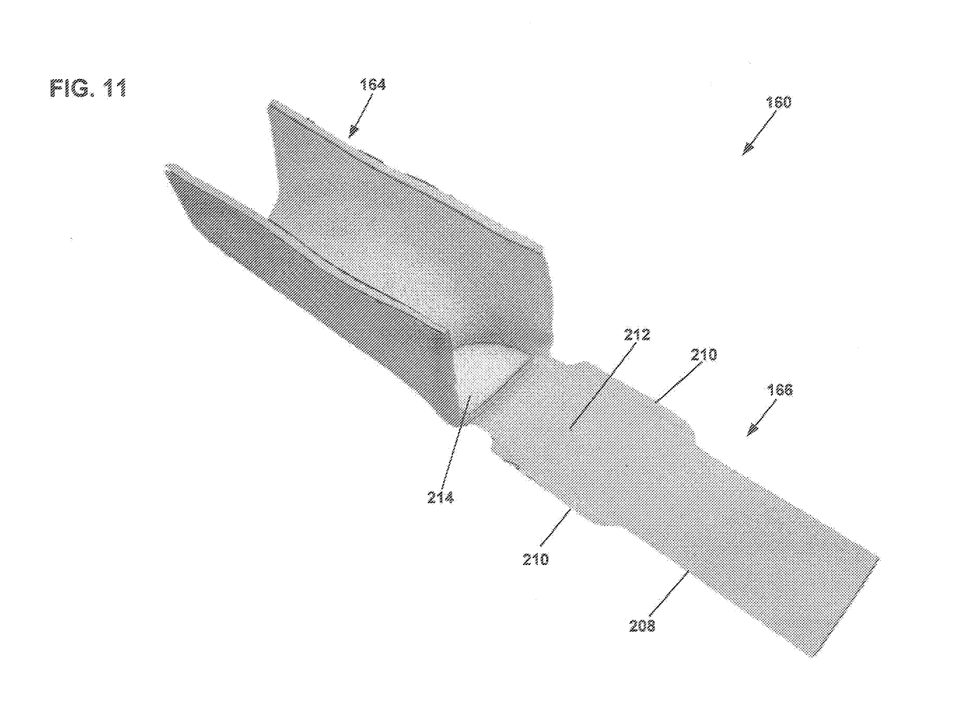

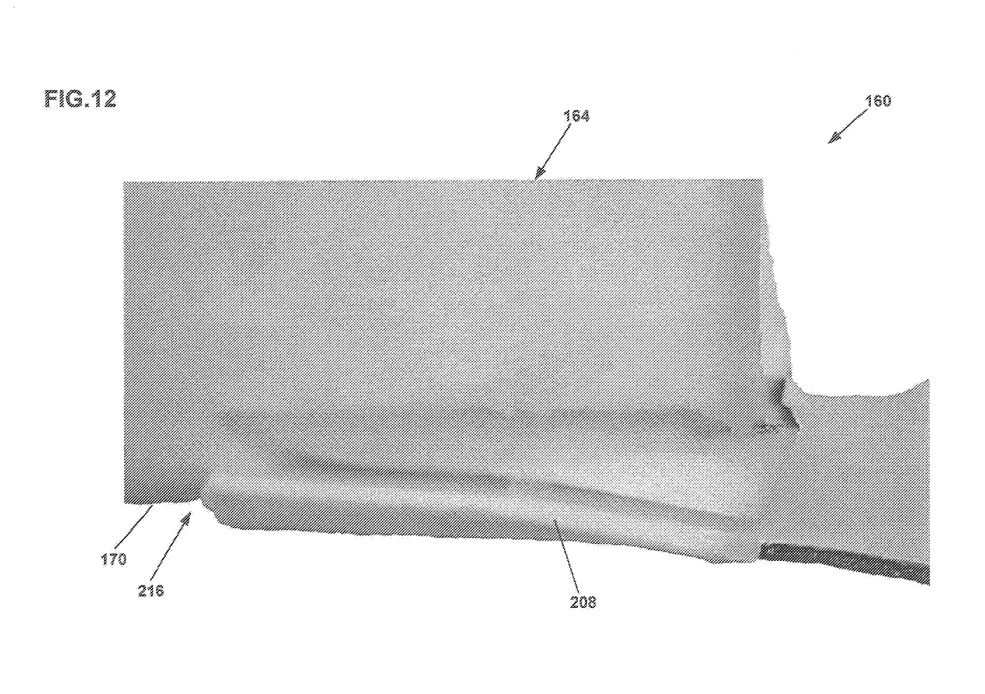

FIG. 6 is a perspective of an example of a heel float cradle 160. In this example, the heel float cradle 160 includes a lower leg support cradle 164 and a foot support platform 166. In this example, the lower leg support cradle 164 and the foot support platform 166 are connected together to form one continuous piece. The heel float cradle 160 can for example be constructed of a variety of materials including elastomers, polyurethane foam, and/or other open and/or closed cell foams or combinations thereof. The durometer and resilience of the material preferably compresses slightly and springs back to shape upon relieving any pressure. The foot support platform 166 is illustrated and described in more detail with reference to FIGS. 11-12.

In this example, the lower leg support cradle 164 includes a posterior flat surface 170 and side walls 172 that extend along the longitudinal sides of the lower leg. The lower leg support cradle 164 provides a longitudinal channel 168 that receives the lower leg of a patient. The posterior flat surface 170 stabilizes the lower leg support cradle 164 and lower leg against rotation when the patient rests in a supine position and the heel float therapeutic boot 100 is supported on a bed. In this example, the lower leg support cradle 164 has a U-shaped cross-section. In other embodiments, the lower leg support cradle 164 can be other cross-sectional shapes, such as, a circular shape.

FIG. 7 is a cross-sectional view of the lower leg support cradle 164 of the heel float cradle 160. As shown, the lower leg support cradle 164 includes an anterior surface 174 along the longitudinal channel 168, a raised surface portion 176, a cutout region 178, and a peripheral edge 180 of the anterior surface 174.

In this example, the anterior surface 174 is contoured and exhibits a compound arcuate shape to support the lower leg or calf. The contoured surface helps to distribute and equalize support on the lower leg or calf and prevents pressure points that might induce skin ulcerations or abrasion. The raised surface portion 176 of the anterior surface 174 is in the region of the Achilles tendon and is shaped to elevate and support the heel in the cutout region 178 with minimal pressure and contact with the boot and away from any support structure, such as a bed, foot stool etc. In this example, the cutout region 178 is an inverted U-shape being adjacent to the peripheral edge of the anterior surface 174 of the lower leg support cradle 164. The cutout region 178 is formed to shelter the suspended heel.

FIG. 8 is a perspective view of the foot support portion 104 of the heel float therapeutic boot 100. In this example, the foot support portion 104 includes a heel tab 182, an overlapping foot member 184, and a toe opening 186. The heel tab 182 and the overlapping foot member 184 are integrally formed and have separate fastening mechanisms. The heel tab 182 can be detached independent of the overlapping foot member 184 either for inspection or ventilation to expose the heel area. In other embodiments, the heel float therapeutic boot 100 may include a heel support structure 308 (FIG. 18) fastened to the foot support portion 104 such that the heel tab 182 may be an optional design. The foot support portion 104 has a length that helps to prevent the foot from popping out underneath or over the top of the foot support portion 104.

In this example, the heel tab 182 has a fastening mechanism 144c that connects the heel tab 182 to the loop material 154 vertically positioned along the lower leg support portion 102 of the heel float therapeutic boot 100. The heel tab 182 is arranged and configured on the left and right side of the heel float therapeutic boot 100. The heel tab 182 is also provided with a hook patch 146 similar to those described for the upper and lower overlapping members 130, 132. Accordingly, the description for the hook patch 146 and the loop patch 148 is hereby incorporated by reference in its entirety for the heel tab 182.

The overlapping foot member 184 includes a left side panel 188 extending from a left side wall 190 of the foot support portion 104 and a right side panel 192 extending from a right side wall 194 of the foot support portion 104. In this example, the left and right side panels 188, 192 are integrally formed with the left and right side walls 190, 194 respectively. In some embodiments, the overlapping foot member 184 has moisture wicking material to absorb moisture and ventilate the foot. A variety of soft, moisture absorbent, air permeable open weave or porous materials can be used. The inside of the overlapping foot member 184 that faces the foot is covered with the fleece material 156 similar to the upper and lower overlapping members 130, 132.

The overlapping foot member 184 has a fastening mechanism 144d that connects the left side panel 188 together with the right side panel 192. The overlapping foot member 184 may also be provided with a hook patch 146 and a loop patch 148 as the fastening mechanism 144d similar to those described for the upper and lower overlapping members 130, 132. Accordingly, many of the concepts and features for the upper and lower overlapping members 130, 132 are similar to the overlapping foot member 184. As such, the description for the hook patch 146 and the loop patch 148 is hereby incorporated by reference in their entirety for the overlapping foot member 184. In some embodiments, the overlapping foot member 184 has a taper configuration that covers a top portion of the foot while leaving an open portion adjacent to the lower leg support portion 102. In other embodiments, the overlapping foot member 184 can cover the entire top of the foot.

In this example, the toe opening 186 is formed by the overlapping foot member 184. The overlapping foot member 184 covers the toes while still providing for ventilation at the toe opening 186.

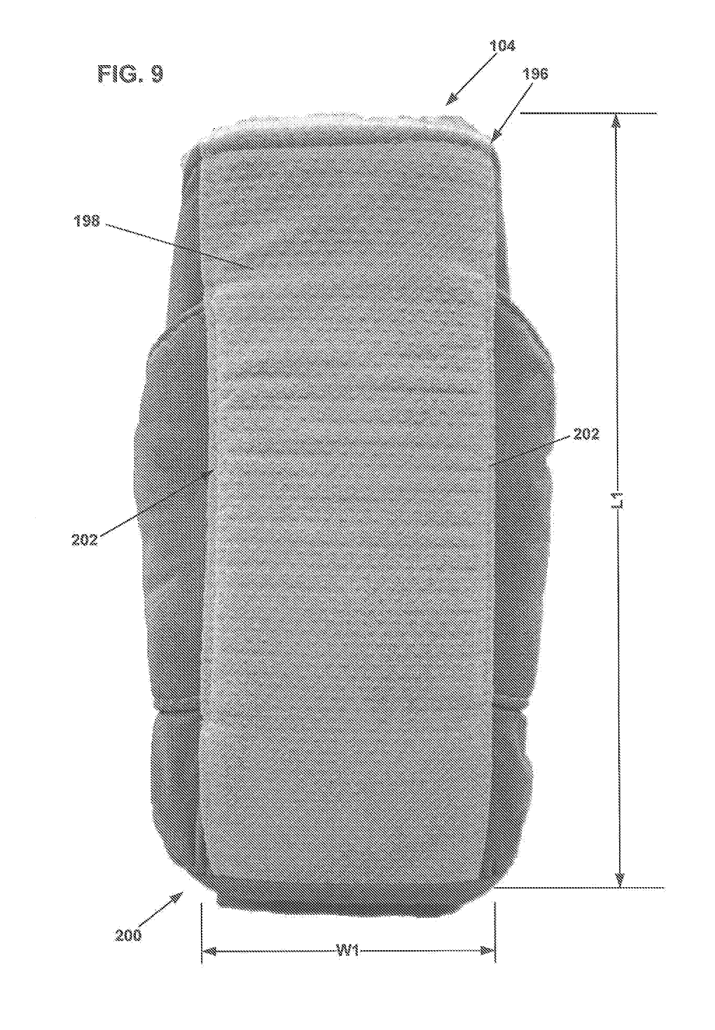

FIG. 9 is a bottom perspective view illustrating exemplary features of the foot support portion 104. In the illustrated example, the foot support portion 104 includes a base 196, a grip material 198, and a flexible hinge 200. The base 196 of the foot support portion 104 has a length L1 and a width W1. In this example, the base 196 of the foot support portion 104 extends beyond the location of the overlapping foot member 184 that covers the toes. The base 196 of the foot support portion 104 extends past the toes and acts as a stub which can help to protect the patient while shuffling along a floor. In other embodiments, the foot support portion 104 may include the heel support structure 308 fastened thereon (see FIG. 18). The heel support structure 308 can help provide added protection to the heel and ankle for reducing pressure thereon when a patient is either shuffling along a floor or lying in bed.

The grip material 198 covers the entire base 196 of the foot support portion 104. The grip material 198 acts as a non-slip material to facilitate safe ambulation over smooth tile or wood floors. The grip material 198 can be constructed of a variety of non-slip materials and is shaped to essentially align with and underlie the base 196 of the foot support portion 104. The grip material 198 includes longitudinal sides 202 that extend the length L1 of the base 196. The grip material 198 is attached to the exterior fabric 162 along the longitudinal sides 202 in the region of the sole. In this example, the grip material 198 is sewn to the exterior fabric 162 of the heel float therapeutic boot 100. Other attachment mechanism may be used, such as, but not limiting to, lamination.

The flexible hinge 200 is formed at the location where the lower leg support portion 102 and the foot support portion 104 are connected together.

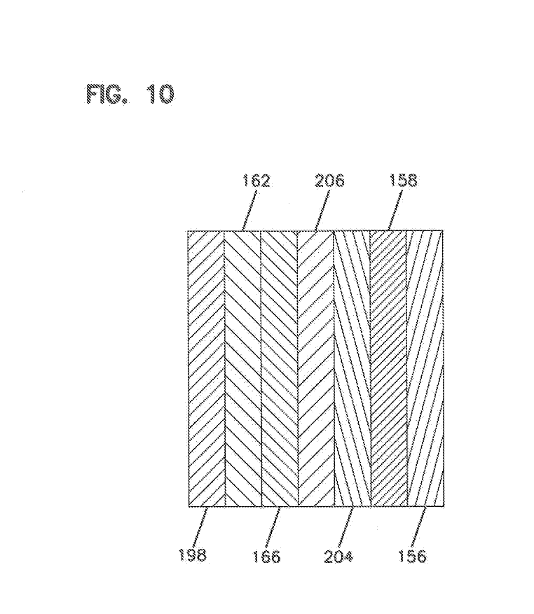

FIG. 10 is a component view of a portion of the foot support portion 104 depicting layers of material therein. In this example, the foot support portion 104 includes a fleece material 156, a lining 158, a foam layer 204, an interior fabric 206, the foot support platform 166, the exterior fabric 162, and the grip material 198. As noted above, the foot support platform 166 is illustrated and described in more detail with reference to FIG. 11. The construction of multiple layers provides a more firm platform for the foot to rest against. The foam layer 204 provides for more cushion to the foot and reduces pressure thereon. The interior fabric 206 is of a type similar to the exterior fabric 162 but rather lined inside portions of the heel float therapeutic boot 100. Many of the concepts and features for these layers have been disclosed or are similar to the lower leg support portion 102 shown in FIG. 5. Accordingly, the descriptions of these layers are hereby incorporated by reference in their entirety for the foot support portion 104.

FIGS. 11-12 illustrate features of the foot support platform 166 of the heel float cradle 160.

FIG. 11 is a perspective view of the foot support platform 166 of the heel float cradle 160. In this example, the foot support platform 166 includes an extended flap portion 208, flaps 210, and apertures 212.

The extended flap portion 208 is arranged and configured to align with the base 196 of the foot support portion 104. In this example, the extended flap portion 208 is constructed of closed cell foam, approximately ⅛-inch thick. In other embodiments, the thickness of the extended flap portion 208 can be greater or less. A proximal end of the extended flap portion 208 can be bonded to a recess 216 (depicted in FIG. 12) formed into the posterior flat surface 170 of the lower leg support cradle 164 adjacent an inverted U-shaped cutout region 178 being adjacent to the peripheral edge of the anterior surface 174 of the lower leg support cradle 164. The lower leg support cradle 164 and the foot support platform 166 can be married together at the recess 216 to create the hinge 200 point in the heel float therapeutic boot 100. The recess 216 can be constructed for receiving other components (e.g., cup) of the heel float therapeutic boot 100. The extended flap portion 208 includes apertures 212 to aerate the foot.

The flaps 210 extend from opposite sides of the extended flap portion 208. The flaps 210 are joined with the other layers in the foot support portion 104. In this example, the wing support structure 106 leverages support from the flaps 210 while being adjustably fastened as desired. The wing support structure 106 is illustrated and described in more detail with reference to FIGS. 14-16. The layers of the foot support portion 104 are attached together along the flaps 210 in the region of the sole. In this example, the layers are sewn together through the flaps 210.

The foam 214 is positioned on the extended flap portion 208 at the proximal end below the raised heel. The foam 214 is below the cutout region 178 and provides the cushion for the patient's heel. In this example, the foam 214 is shaped as a half moon. In other embodiments, the foam 214 can take the form of other shapes, such as, circles, square, or rectangular etc.

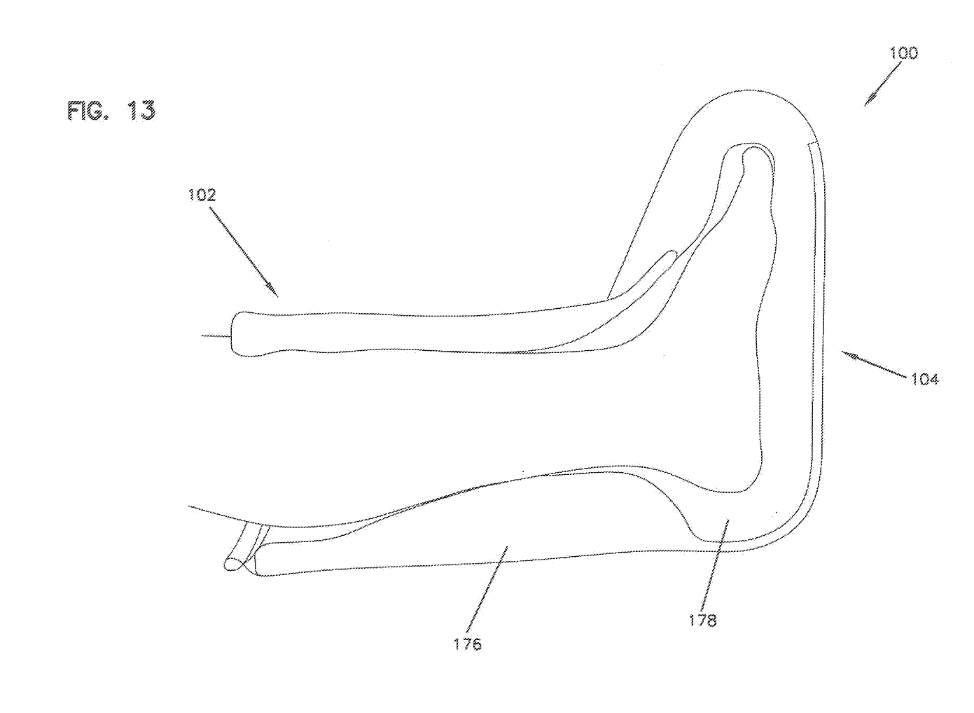

FIG. 13 depicts a cross-sectional view of an example of a patient's foot in the heel float therapeutic boot 100. In this example, the heel is shown elevated with the foot positioned upright at approximately 90 degrees.

FIG. 14 is a side perspective view of an example of the wing support structure 106 attached to a heel float therapeutic boot 100. The wing support structure 106 of the heel float therapeutic boot 100 supports the foot in a preferred position. The wing support structure 106 is attached to the heel float therapeutic boot 100 and is configured to be adjustable to obtain the preferred position. The arrangement and configuration of the back side 228 of the wing support structure 106 provides for flexibility of attaching the wing support structure 106 to the loop material 154 that helps place the foot support portion 104 in the proper position. The wing support structure 106 can be placed anywhere along the loop material 154 as desired. The positioning of the wing support structure 106 will vary with each patient. A proper fit can be obtained for any patient from a pediatric size foot up to a man's size 14 foot. The configuration of the wing support structure 106 also provides for easy access to the heel of the foot.

The wing support structure 106 is adjustable to be positioned along the loop material 154. In this example, the wing support structure 106 is adjustable along the lower leg support portion 102 perpendicular to Axis A. Axis A extends longitudinally through the foot support portion 104. The wing support structure 106 has the flexibility to position the foot between 75-110 degrees about axis A. This feature provides for a patient to have the ability to access any part of the bottom of the foot and position the foot as desired. The width W2 of the bottom side 22 of the wing support structure 106 and the configuration of attachment not only helps to keep the foot in an upright position, but it also helps to keep the foot in place so that it is less likely to rotate or migrate around inside of the heel float therapeutic boot 100.

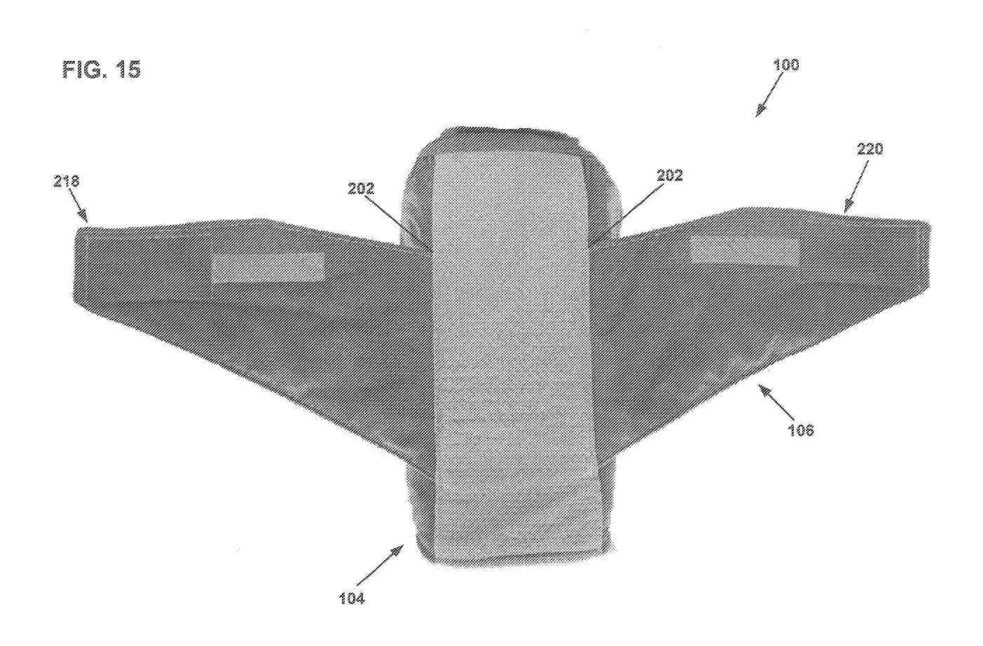

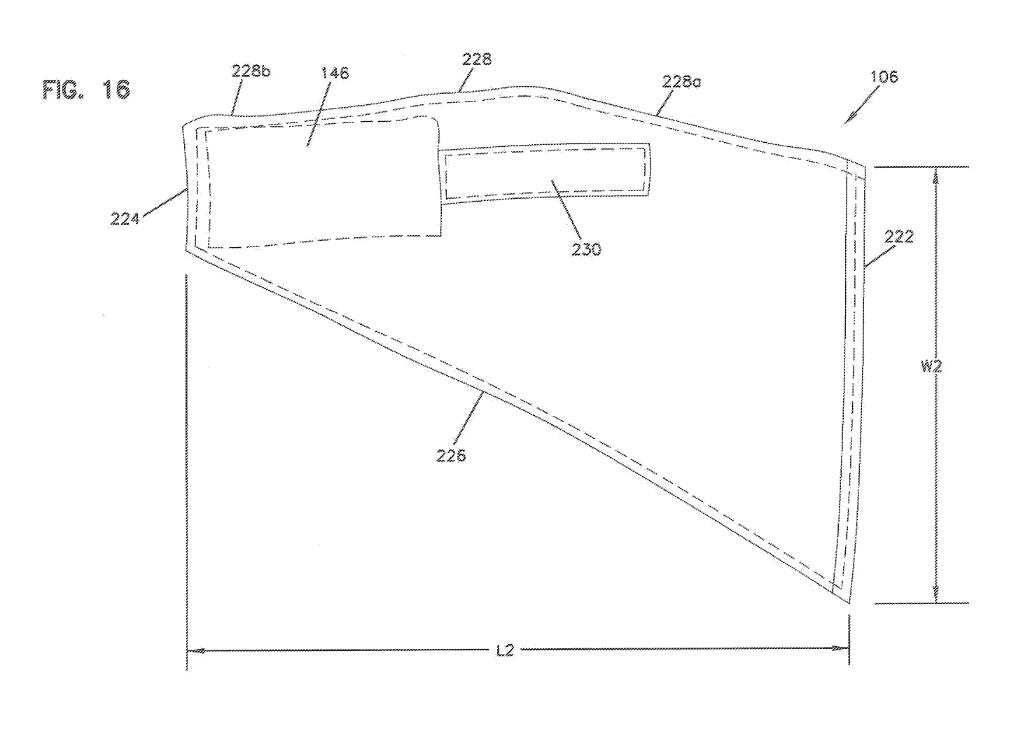

FIGS. 15-16 illustrate features of the wing support structure 106.

FIGS. 15-16 are perspective views of the wing support structure 106.

Referring to FIG. 15, the bottom side 222 of the wing support structure 106 is sewn along the longitudinal sides 202 and intersects together with the layers in the foot support portion 104. Portions of the wing support structure 106 extend beyond the sewn area along the longitudinal sides 202 to help prevent the wing support structure 106 from detaching from the foot support portion 104. The bottom side 222 of the wing support structure 106 has a width W2 that expands along a portion of the base 196 of the foot support portion 104. The bottom side 222 of the wing support structure 106 is positioned mostly centered on the base 196. The configuration of the wing support structure 106 eliminates the problem of generating pressure points underneath the foot because no portion of the wing support structure 106 goes across the ball of the foot. The wing support structure 106 is anchored to the sole and helps to spread the pressure points or force across the patient's foot while holding the foot support portion 104 of the heel float therapeutic boot 100 in a desired position. The wing support structure 106 is arranged and configured to keep the foot support portion 104 of the heel float therapeutic boot 100 pulled up or upright.

In the illustrated example, the wing support structure 106 includes a left support wing 218 and a right support wing 220. The left and right support wings 218, 220 are arranged and configured on opposite sides of the heel float therapeutic boot 100. The left and right support wings 218, 220 are each attached to the heel float therapeutic boot 100 along the longitudinal sides 202 in the region of the sole. In the illustrated example, the left and right support wings 218, 220 are permanently fastened to the foot support portion 104. The configuration provides for a wing support structure 106 that is non-adjustable in relation to the foot support portion 104. The wing support structure 106 is adjustably fastenable along the sides of the lower leg support portion 102 (shown in FIG. 14). The wing support structure 106 is wide enough and fixed to the foot support portion 104 to help prevent the foot of a patient from wrapping across the top of the heel float therapeutic boot 100 or popping out underneath the heel float therapeutic boot 100. The wing support structure 106 has a wide area to help disperse any points of pressure, which lowers the likelihood of having a pressure area anywhere in the foot region. Having the wing support structure 106 permanently attached at the sole not only helps to prevent pressure points on the foot but it also helps to eliminate improper positioning of the strap over the foot. In other embodiments, the heel support structure 308 can be attached to the heel float therapeutic boot 100 to help prevent pressure points on the foot and to help position the foot properly. It is to be understood that various combinations of features may be used with the heel float therapeutic boot 100. For example in other embodiments, the heel float therapeutic boot 100 may include only the heel support structure 308 and not the wing support structure 106.

Referring to FIG. 16, the wing support structure 106 includes a bottom side 222, an opposing top side 224, a front side 226, a back side 228, and a loop stripe 230. The wing support structure 106 further includes a length L2 and a width W2.

In the illustrated example, the front side 226 of the wing support structure 106 is angled relative to the back side 228 of the wing support structure 106 such that the top side 224 of the wing support structure 106 is narrower than the bottom side 222 of the wing support structure 106. The front side 226 of the wing support structure 106 being about 9 to 11 inches in length. The top side 224 of the wing support structure 106 includes a hook patch 146 as already described above. The top side 224 is attached to the loop material 154 to position the wing support structure 106 as desired. The top side 224 of the wing support structure 106 being about 1 to 2 inches in length.

In the illustrated example, the back side 228 of the wing support structure 106 is about 9 to 10 inches in length. The back side 228 has an angled portion 228a parallel to the front side 226 of the wing support structure 106. The angled portion 228a is about 4 to 5 inches in length. The angled portion 228a extends from the bottom side 222 of the wing support structure 106. The angled portion 228a forms a perpendicular portion 228b that is perpendicular to the bottom side 222 of the wing support structure 106. The perpendicular portion 228b being about 4 to 5 inches in length. In other embodiments, other shapes and configurations are possible. The left and right support wings 218, 220 are shaped expanding a length and width with respect to the heel float therapeutic boot 100. In other embodiments, other shapes and configurations are possible.

The left and right support wings 218, 220 each include moisture wicking material similar to the overlapping foot member 184. The left and right support wings 218, 220 each have a fastening mechanism 144e. As illustrated in FIG. 14, the left and right support wings 218, 220 are provided with the hook patch 146 and the loop material 154 as the fastening mechanism 144e similar to those described above. As such, the description for the hook patch 146 and the loop material 154 is hereby incorporated by reference in their entirety for the left and right support wings 218, 220.

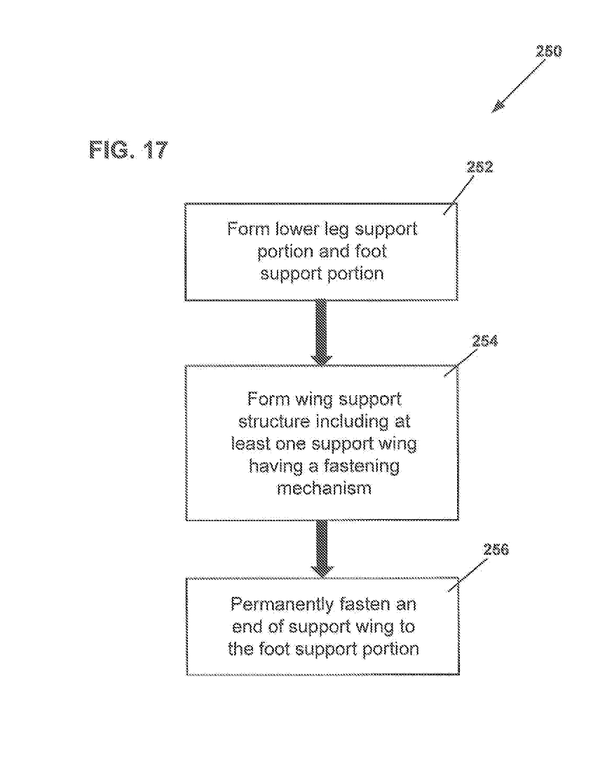

FIG. 17 is a flow chart illustrating an example method 250 of making a heel float therapeutic boot 100. In this example, the method 250 includes operations 252, 254, and 256.

The operation 252 is performed to form a lower leg support portion 102 and a foot support portion 104. Examples of the lower leg support portion 102 and the foot support portion 104 are shown and described with reference to FIGS. 1-3. In some embodiments, the foot support portion 104 includes a foot support platform 166. An example of a foot support platform 166 is shown and described with reference to FIG. 11.

The operation 254 is performed to form the wing support structure 106. In some embodiments, the wing support structure 106 includes at least one support wing 106(A,B). In some embodiments, the wing support structure includes a left support wing 106A and a right support wing 106B. The support wings 106 include a first end portion and a second end portion. The second end portion is configured to be adjustably fastened to opposite sides of the lower leg support portion. Examples of the wing support structure 106 and support wings 106A and 106B are illustrated and described in more detail in FIGS. 14-16.

The operation 256 is performed to permanently fasten the first end portion of the support wing 106(A,B) to the foot support portion 104. In some embodiments, operation 256 includes permanently fastening the first end portions of the left and right support wings 106A and 106B to the foot support portion 104. In some embodiments, the one or more support wings 106 are permanently fastened to the foot support platform 166. An example of the fastening operation is sewing with thread. Other fastening operations can also be used, such as described herein. Examples showing the attachment of the wing support structure 106 and support wings 106A and 106B to the foot support portion 104 are shown in FIGS. 1 and 14.

After the heel float therapeutic boot 100 has been made, a method of using the heal float therapeutic boot 100 can be performed. In one example embodiment, the heel float therapeutic boot 100 is arranged on a foot and lower leg of a patient, such as illustrated in FIG. 13. The foot and lower leg are then secured in the heel float therapeutic boot 100 using the various panels and fasteners described herein. Additionally, the second end of one or more of the support wings 106A and 106B are adjustably secured to sides of the lower leg support portion 102. The heal float therapeutic boot 100 securely supports the foot and lower leg in a desired position.

FIGS. 18-19 illustrate features of the heel support structure 308.

FIG. 18 is a side perspective view of another example of a heel float therapeutic boot 300 with a heel support structure 308 fastened thereon by a fastener, such as, but not limited to, a rivet 310. Other fastening devices and arrangements can be used in accordance with the principles disclosed. The heel float therapeutic boot 300 includes a lower leg support portion 302, a foot support portion 304, and a wing support structure 306. Many of the concepts and features for these structures have been disclosed or are similar to the heel float therapeutic boot 100 shown in FIGS. 1-17. Accordingly, the descriptions and features of these structures are hereby incorporated by reference in their entirety for the heel float therapeutic boot 300. In this example, the heel support structure 308 extends a height H1 along the lower leg support portion 302 and a length L3 along the foot support portion 304.

In some embodiments, the heel support structure 308 helps provide rigidity to the heel float therapeutic boot 300 to prevent foot drop by keeping the foot in an upright position. At other times, there are instances where patients may bend their knees while lying in bed or the patient may decide to lie on one side while in the bed. In these situations, the heel support structure 308 can help reduce any pressure that may result when the heel float therapeutic boot 300 is pressed on the bed. The heel support structure 308 can be helpful for patients who are not only bed ridden, but are in wheel-chairs. For example, the heel support structure 308 can provide additional support for patients who at times use the ball or heel of their foot to walk across the floor while seated in the wheel-chair. The heel support structure 308 can provide a rigid surface that helps protect the heel from pressure points that may result from walking on the heels or from similar activities.

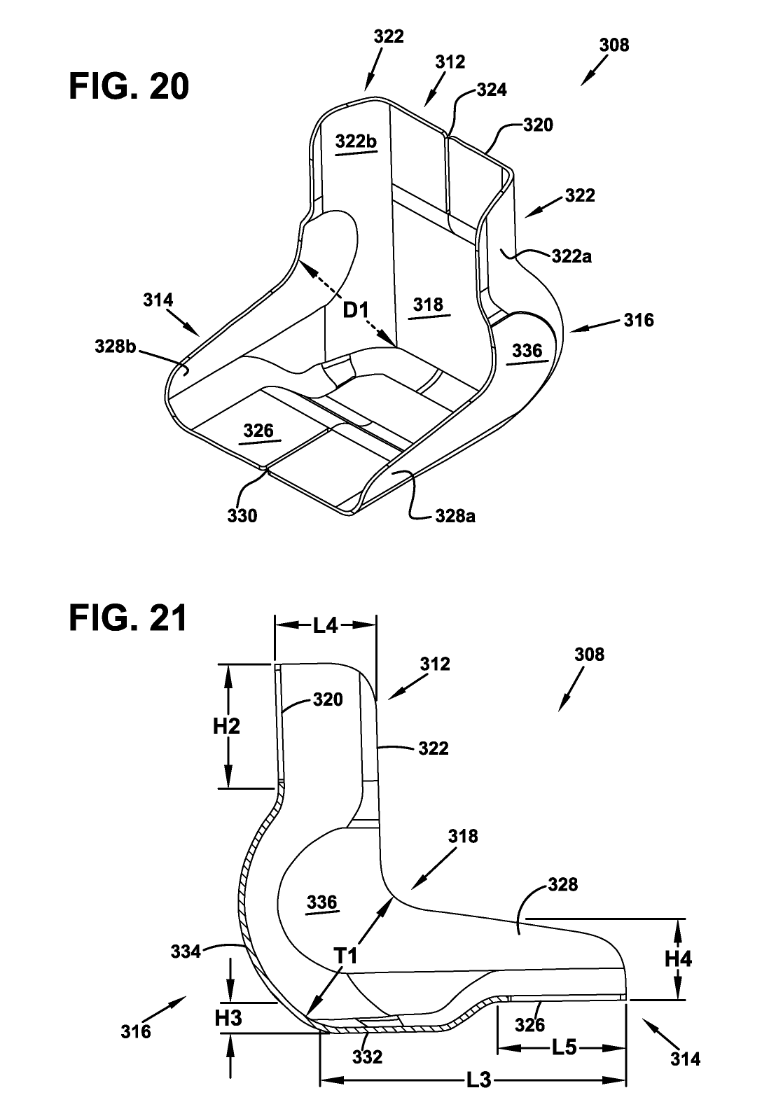

Referring to FIG. 19, the heel support structure 308 typically covers the heel of a patient's foot and ankle to help protect against pressure ulcers formed on the skin by providing a rigid support structure to help keep the foot elevated in the heel float therapeutic boot 300. The heel support structure 308 is illustrated and described in more detail with reference to FIGS. 20-23.

FIGS. 20-23 illustrate features of the heel support structure 308.

FIGS. 20-23 are perspective front, side, top, and bottom views of the heel support structure 308. In this example, the heel support structure 308 includes a first end 312, a second end 314, and a center 316 integrated with or coupled to the first and second ends 312, 314. As shown, the heel support structure 308 is formed of one piece to define a cup like pocket 318 around the heel float therapeutic boot 300.

The heel support structure 308 is arranged and configured to attach to the heel float therapeutic boot 300 at the lower leg support portion 302 and the foot support portion 304. In this example, the rivets 310 fasten the heel support structure 308 to the exterior side of both the lower leg support portion 302 and the foot support portion 304 of the heel float therapeutic boot 300. The heel support structure 308 maybe covered on the exterior surface with a fabric or foam to prevent injury to other limbs while lying down in bed. The heel support structure 308 may also include a foam or fabric on the interior side to help maintain or increase blood flow. The increase in blood circulation can minimize the potential of developing wounds or sores on the skin. In other embodiments, the heel support structure 308 can be arranged in the interior side of the heel float therapeutic boot 300. For example, the heel support structure 308 can be placed between any of the layers of materials in the lower leg support portion 302 or the foot support portion 304. In other embodiments, the heel support structure 308 can be integrally formed with a heel float cradle.

In this example, the first end 312 has a first wall 320 and flaps 322 extending from the first wall 320. The flaps 322 are integral with or coupled to the first wall 320 to form one piece. The first end 312 is constructed to fit over the lower leg support portion 302 of the heel float therapeutic boot 300. The first end 312 extends up the lower leg support portion 302 having a height H2 to help support the patient's foot in an upright position.

The first wall 320 defines an elongated slot 324 for receiving the rivet 310 for attachment of the heel support structure 308 to the heel float therapeutic boot 300. The elongated slot 324 provides flexibility for placement of the rivet 310 to obtain the best support. The first wall 320 is similarly dimensioned with the lower leg support portion 302 such that they can be properly aligned and coupled together. The first wall 320 has a width W3 such that the elongated slot 324 is positioned in the middle of the first wall 320. It is to be understood that the geometry and size of the first wall 320 can vary with other embodiments.

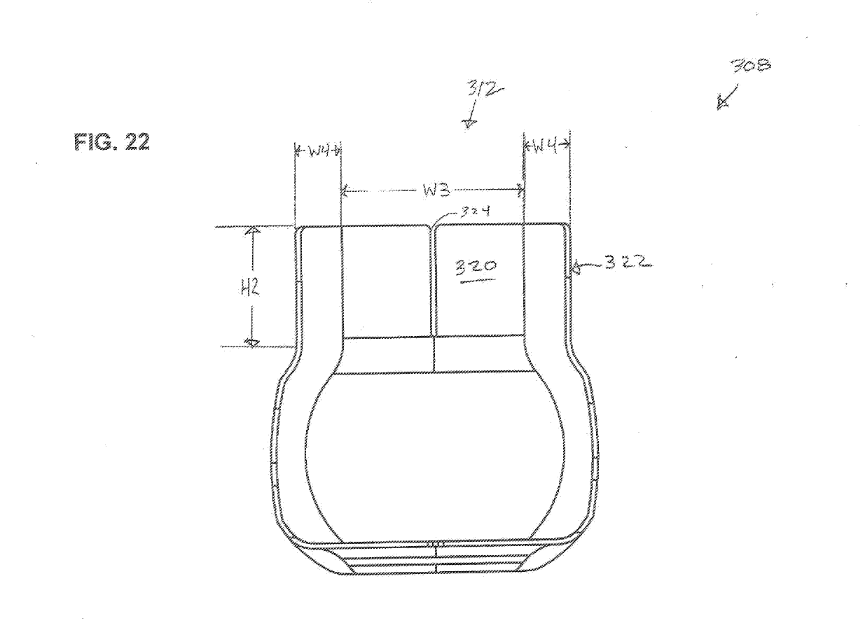

The flaps 322 of the first end 312 include a left flap 322a and a right flap 322b. Referring to FIG. 22, the flaps 322 curve outwardly from the first wall 320 and have a width W4 that extends beyond the width W3 of the first wall 320. The flaps 322 have a length L4 extending perpendicularly from the first wall 320. The length L4 of the flaps 322 can vary with other embodiments. The length L4 of the flaps 322 is constructed to be a sufficient distance from the first wall 320 in order to help support and protect the lower leg and sides of the ankle while the foot is in an upright position or lying on its side. In this example, the flaps 322 add a rigid support structure such that they function as a shield to help protect the patient from pressure sores.

In this example, the second end 314 has a second wall 326 and panels 328 extending from the second wall 326. The panels 328 are integral with or coupled to the second wall 326 to form one piece. The second end 314 is constructed to fit over the foot support portion 304 of the heel float therapeutic boot 300. The second end 314 is sized similarly to the heel float therapeutic boot 300 such that the two can be attached together. Because of the multiple layers of the heel float therapeutic boot 300 and the fabric or foam material on the heel support structure 308, pressure points can be minimized.

The second wall 326 can extend along the foot support portion 304 having a length L5. In this example, the second wall 326 extends from about the arch of a foot of the patient to about a ball of the foot. The second wall 326 defines an elongated slot 330 for receiving the rivet 310 for attachment of the heel support structure 308 to the heel float therapeutic boot 300. The elongated slot 330 provides flexibility for placement of the rivet 310 to obtain the best support for the patient.

In this example, the second end 314 further includes a heel portion 332. The heel portion 332 helps to provide the patient with a more rigid support structure that can allow the patient to stand or walk short distances with more stability and protection. In one example, patients walking to the restroom may find the heel portion 332 helpful to prevent any pressure points on the heel. Again, patients in a wheel chair may find the heel portion 332 helpful to prevent pressure points on the surface of the heel when the heel is used to walk across the floor. The heel portion 332 can be integrated with or coupled to the second wall 326 to form one piece. The heel portion 332 can have a height H3 that extends below the second wall 326. In some embodiments, the bottom surface of the heel portion 332 can have a non-slip surface to facilitate safe ambulation over smooth tile or wood floors. The non-slip surface may be formed by including non-slip material (i.e. grip material, abrasive material) on the heel portion 332. The non-slip material can be constructed of a variety non-slip material and shaped to essentially align with and underlie the second end 314 of the heel support structure 308. In other embodiments, the non-slip material may cover the first and second ends 312, 314 of the heel support structure 308. In some embodiments, the non-slip surface may be constructed by providing plastic pegs on the heel support structure 308 to help provide a non-slip surface. Other non-slip materials may be used in accordance with this disclosure.

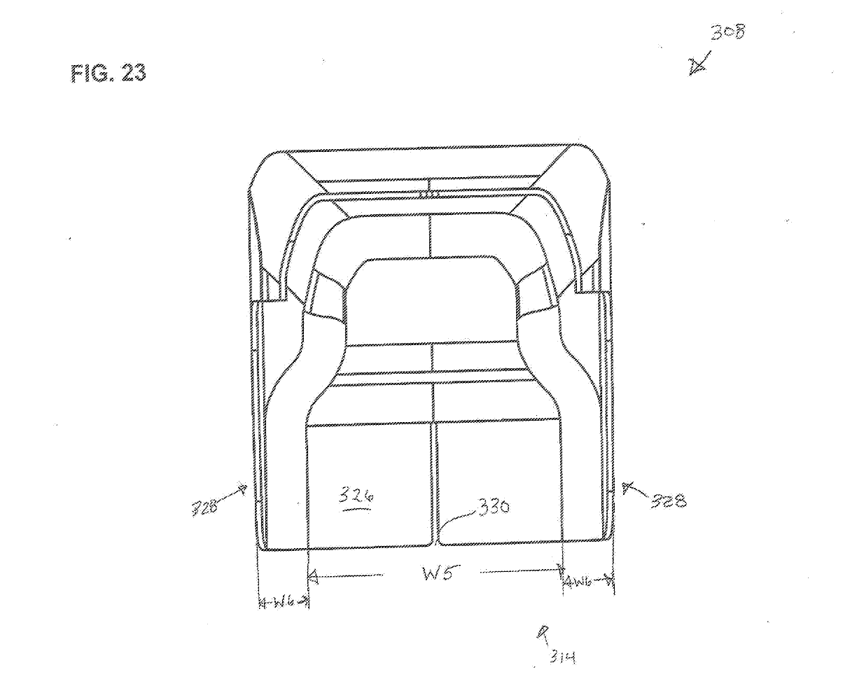

The length L3 of the second end 214 helps to distribute and equalize support on the lower leg or calf and helps prevent pressure points that might induce skin ulcerations or abrasion. It is to be understood that the size and configuration of the heel portion 332 of the second end 314 may vary. The second wall 326 is similarly dimensioned with the foot support portion 304 such that they can be properly aligned and coupled together. Turning to FIG. 23, the second wall 326 has a width W5 such that the elongated slot 330 is positioned in the middle of the second wall 326. It is to be understood that the geometry and size of the second wall 326 of the second end 314 may vary with other embodiments.

The panels 328 of the second end 314 include a left panel 328a and a right panel 328b. In this example, the panels 328 curve outwardly from the second wall 326 and have a width W6 that extends beyond the width W5 of the second wall 326. The panels 328 have a variable up to height H4 extending perpendicularly from the second wall 326. The height H4 of the panels 328 can vary with other embodiments. The height H4 of the panels 328 extend a distance from the second wall 326 in order to help support and protect the foot and sides of the foot while it is in an upright position or lying on its side. In this example, the panels 328 add a rigid support structure that allows it to function as a shield to help protect the patient from pressure sores.

The center 316 of the heel support structure 308 defines the pocket 318 in which the heel of the patient is suspended above to help protect it from pressure points. In this example, the center 316 includes a hub 334 and corners 336 extending from opposites sides of the hub 334. The corners 336 are integral with or coupled to the hub 334 to form one piece. The hub 334 is in the region of the Achilles tendon and is shaped to elevate and support the heel of the patient in the pocket 318 with minimal pressure and contact with the boot and away from any support structure, such as a bed, foot stool etc. In this example, the pocket 318 is a cup-shape arranged and configured adjacent to the corners 336. The pocket 318 is formed to shelter the suspended heel of the patient. The pocket 318 can have a depth distance D1 (FIG. 20) to receive the heel of a patient wearing a heel float therapeutic boot 300.

In this example, the corners 336 of the center 316 have a bubble like configuration to help provide shelter to the ankle. The corners 336 can have a thickness of T1 (FIG. 21) to help protect the heel of the patient and minimize contact between the heel float therapeutic boot 300 and any support structure while being worn. The corners 336 are integral with and coupled to the flaps 322 and panels 328 of the heel support structure 308. The geometry and configuration of the corners 336 may vary in other embodiments.

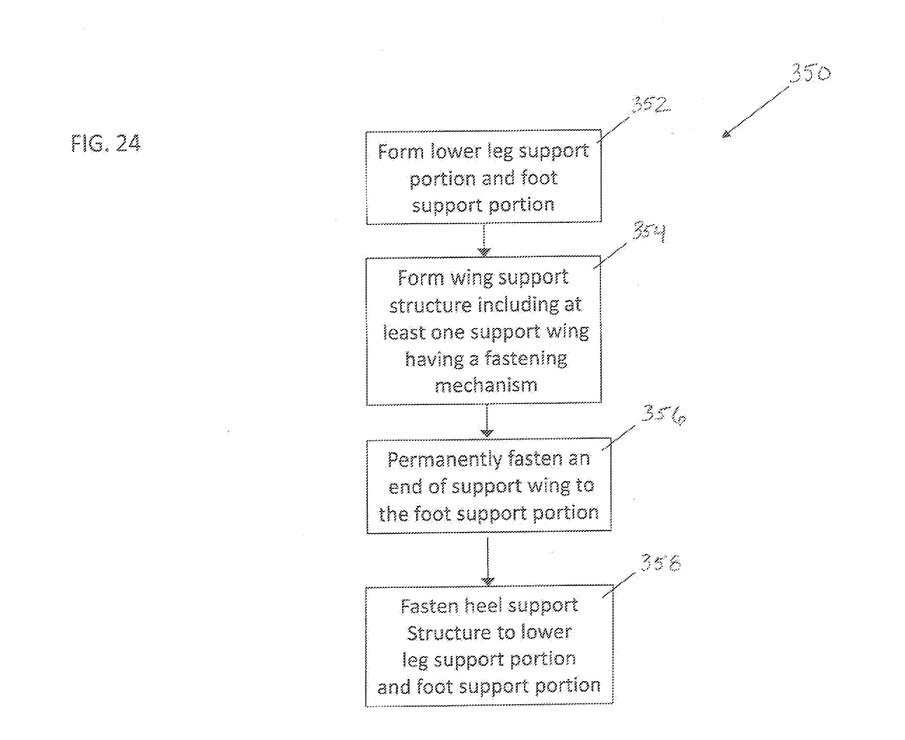

FIG. 24 is a flow chart illustrating an example method 350 of making a heel float therapeutic footwear apparatus 300. In this example, the method 350 includes operations 352, 354, 356, and 358. The operations 352, 354, and 356 are similar to those methods described for operations 252, 254, and 256. Accordingly, the description for the operations 252, 254, and 256 is hereby incorporated by reference in its entirety for the operations 352, 354, and 356.

The operation 358 is performed to fasten a heel support structure 308 on the lower leg support portion 302 and foot support portion 304 of a boot. Examples of the heel support structure 308 are shown and described in reference to FIGS. 18-23.

After the heel float therapeutic footwear apparatus 300 has been made, a method of using the heal float therapeutic footwear apparatus 300 can be performed. In one example embodiment, the heel float therapeutic footwear apparatus 300 is arranged on a foot and lower leg of a patient, such as illustrated in FIG. 19. The foot and lower leg are then secured in the heel float therapeutic footwear apparatus 300 using the various panels and fasteners described herein. Additionally, the heel support structure 308 is adjustably secured to the lower leg support portion 302 and the foot support portion 304. The heel support structure 308 provides rigidity and protection while supporting the foot and lower leg in a desired position.

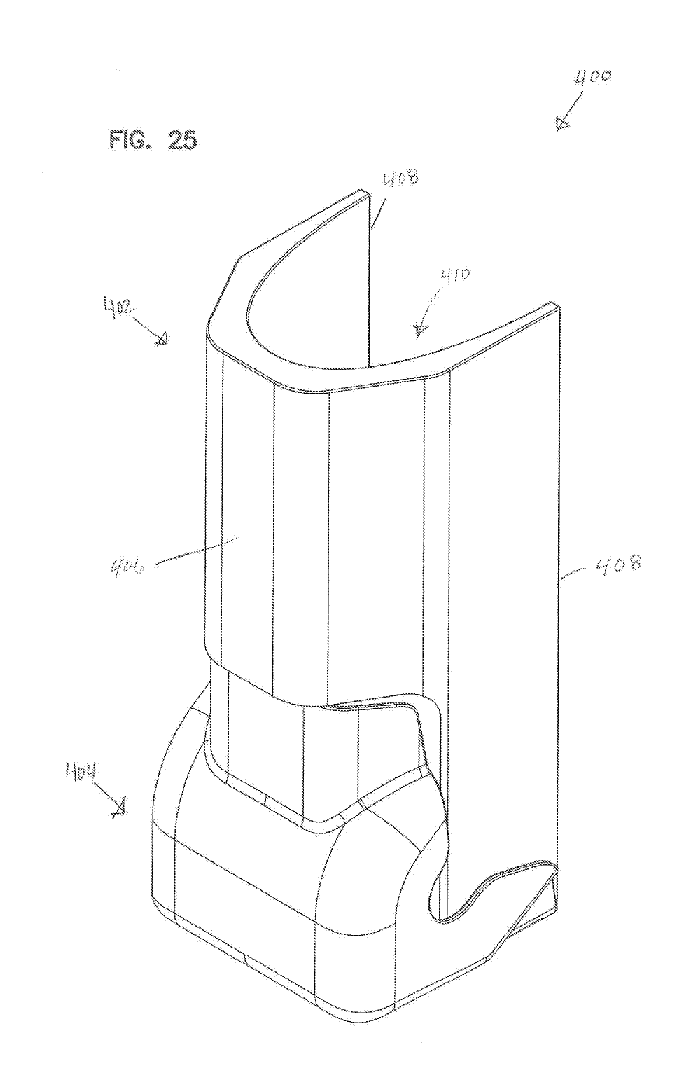

FIGS. 25-27 illustrate an example therapeutic boot support 400 including another example of a heel float cradle 402 and a heel support structure 404. Many of the possible aspects and features of the heel float cradle 160 and the heel support structure 308 discussed above are applicable to the heel float cradle 402 and the heel support structure 404 described below as well.

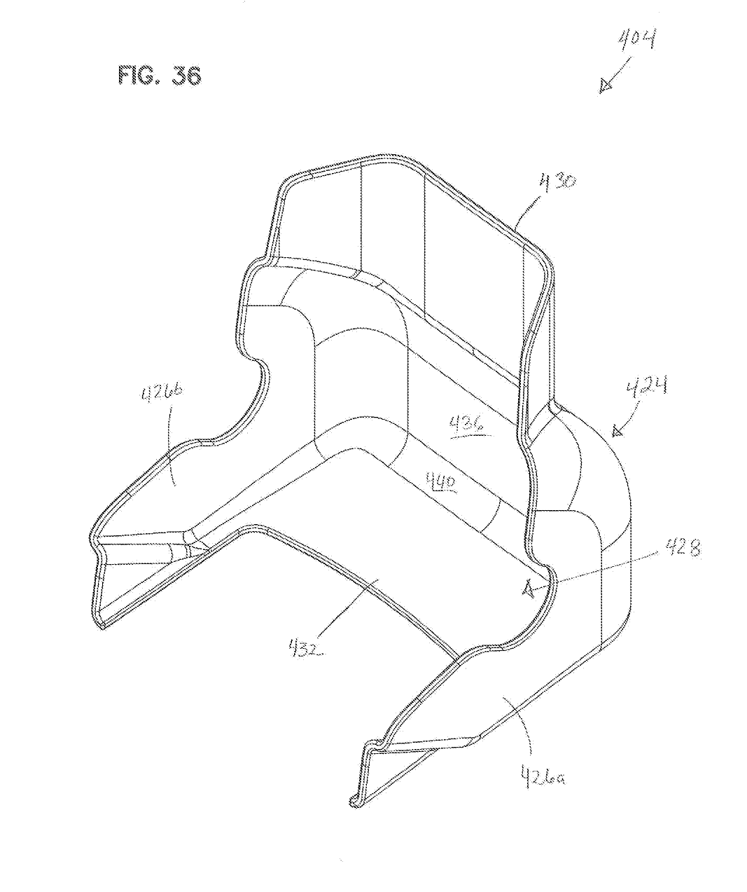

FIG. 25 is a perspective view of the therapeutic boot support 400 having the heel float cradle 402 and the heel support structure 404 attached together. The heel support structure 404 can be attached to the heel float cradle 402 by adhesive. In other examples, the heel support structure 404 can be attached to the heel float cradle 402 by other attachment mechanisms, such as, fasteners, sewing, or combinations of these or other attachment means. The heel float cradle 402 can for example be constructed of a variety of materials including elastomers, polyurethane foam, and/or other open and/or closed cell foams or combinations thereof. The durometer and resilience of the material preferably compresses slightly and springs back to shape upon relieving any pressure. The heel support structure 404 is illustrated and described in more detail with reference to FIGS. 36-42.

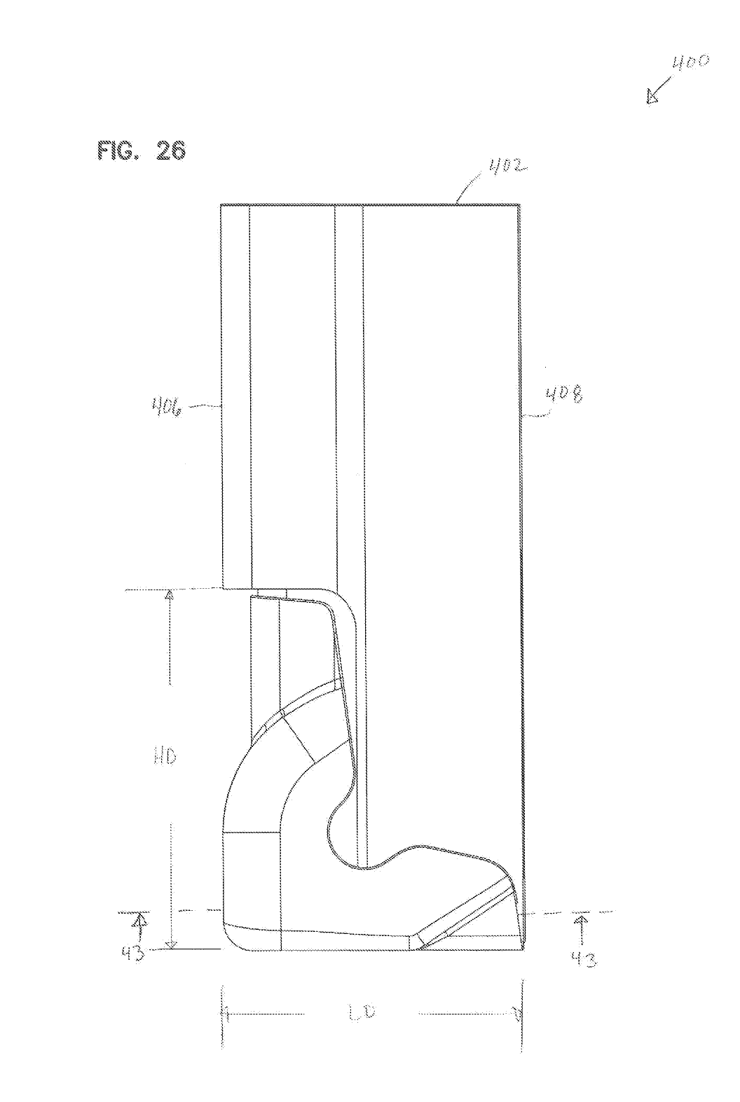

FIG. 26 is a side perspective view of the example therapeutic boot support 400. In this example, the heel support structure 404 can extend a height distance HD and a length distance LD along the heel float cradle 402.

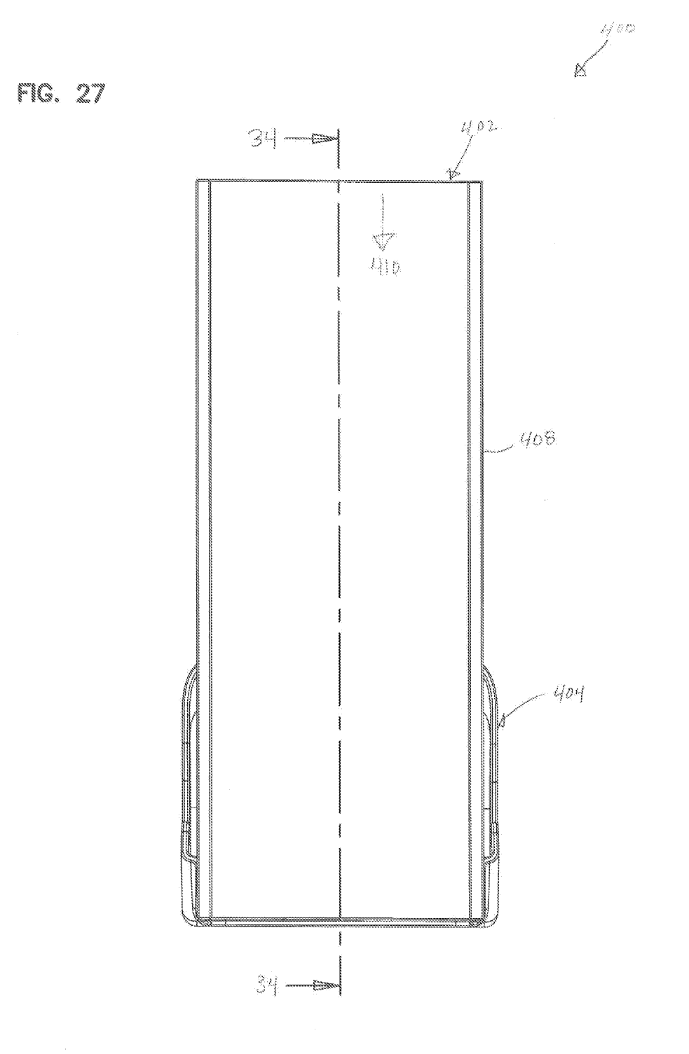

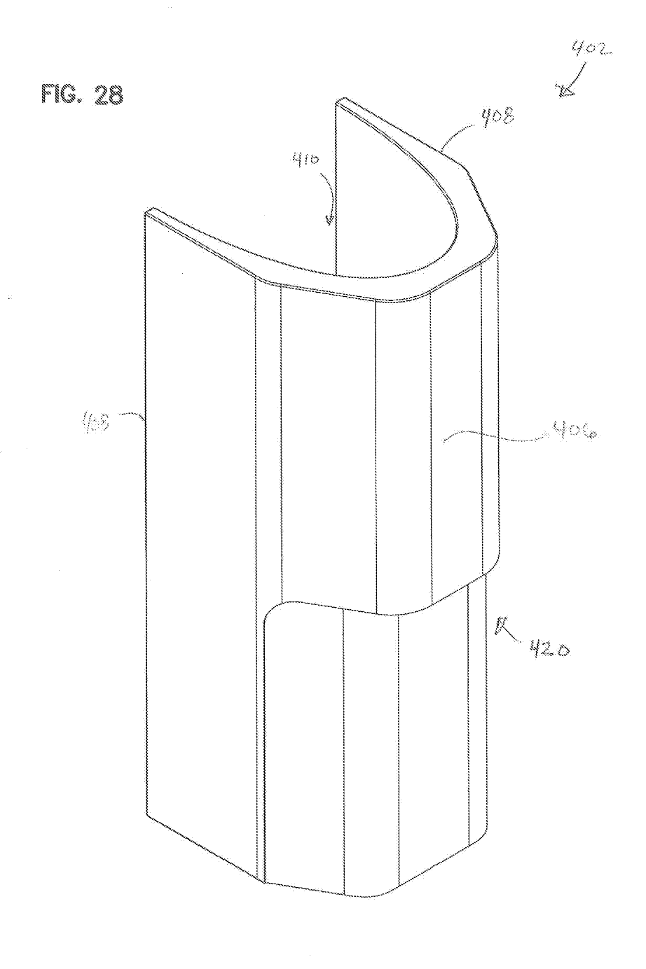

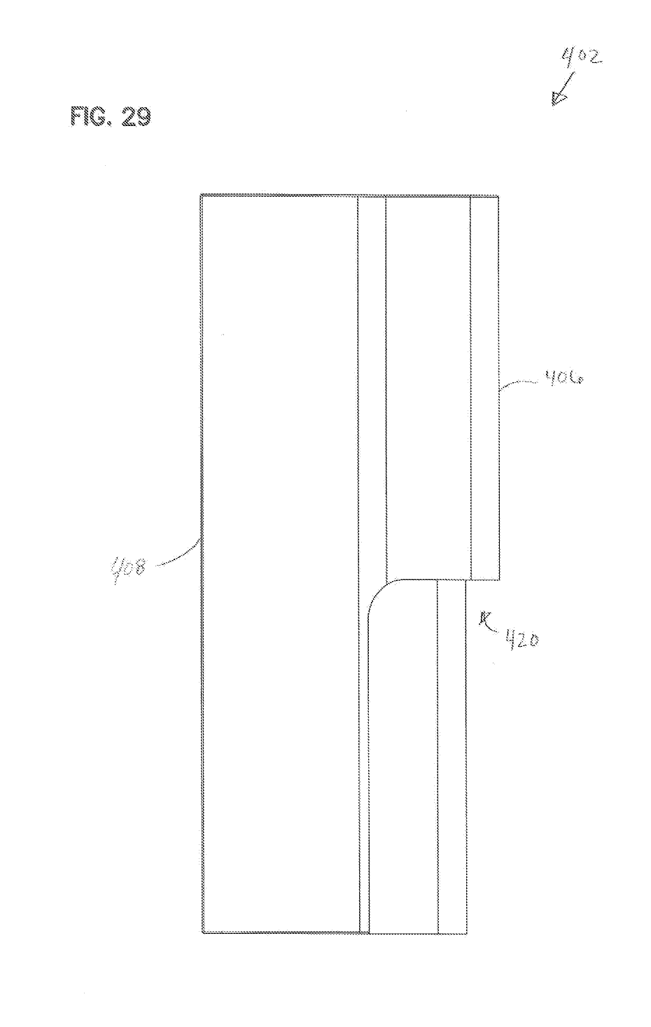

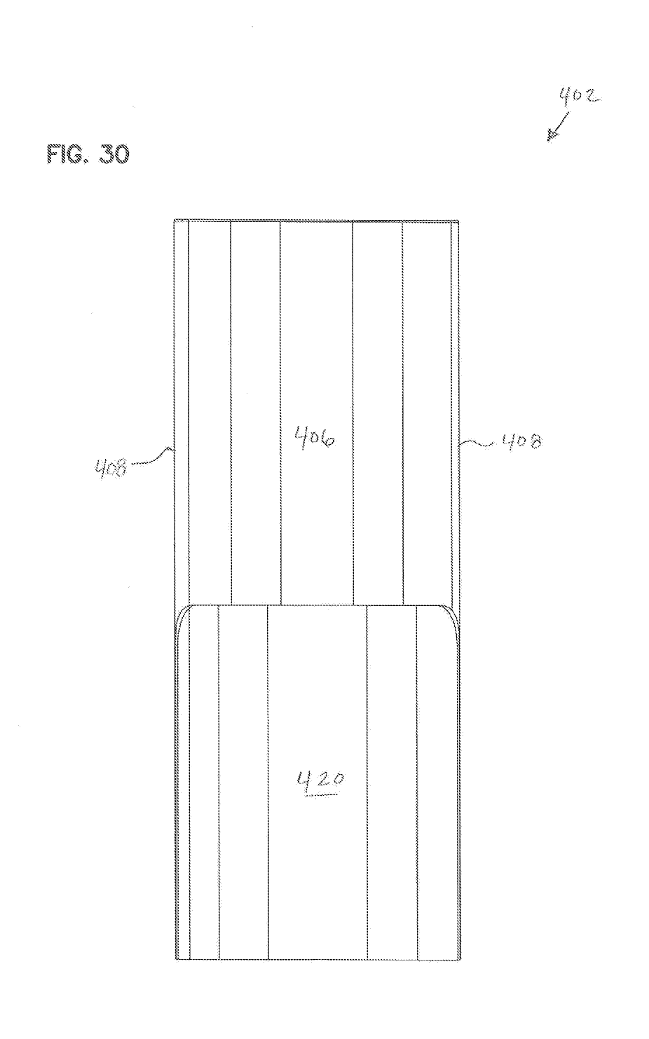

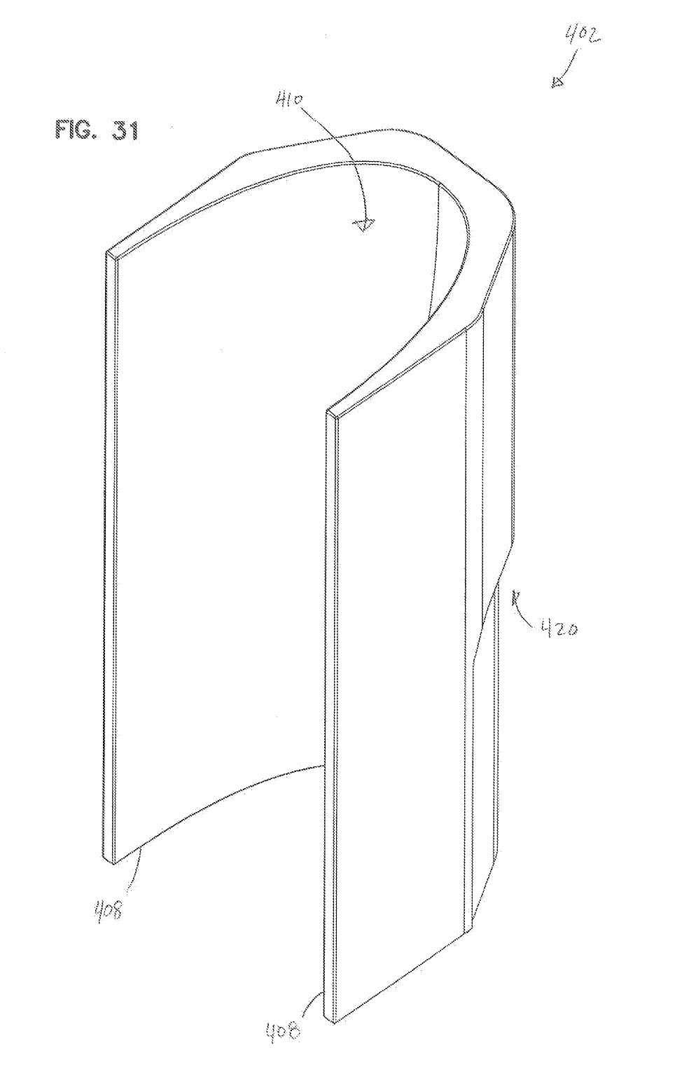

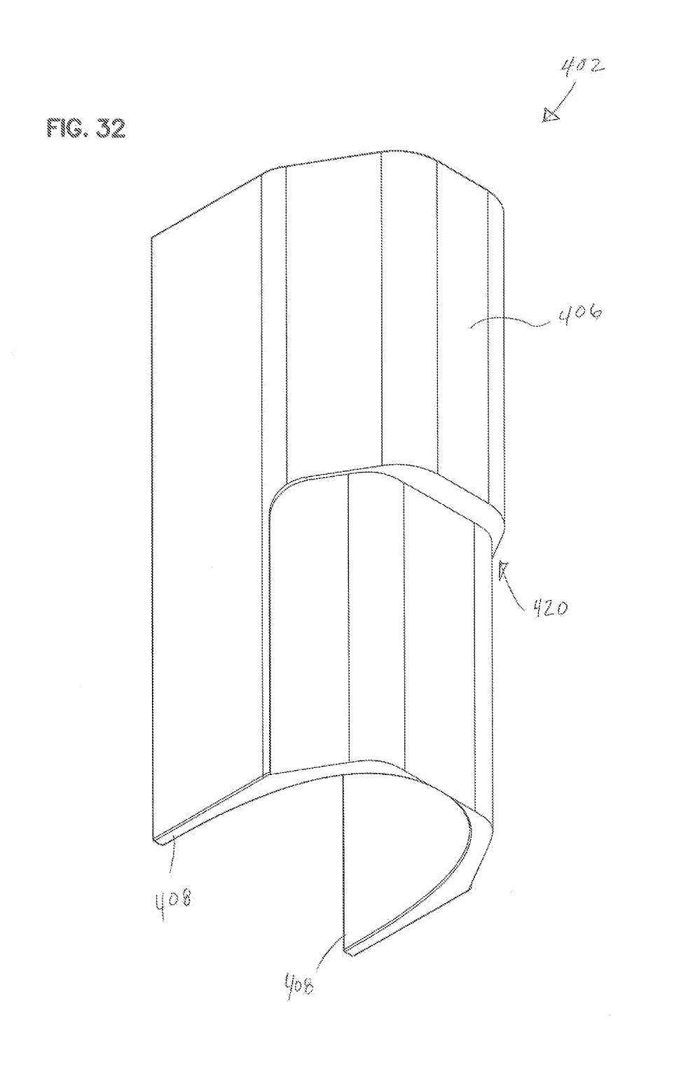

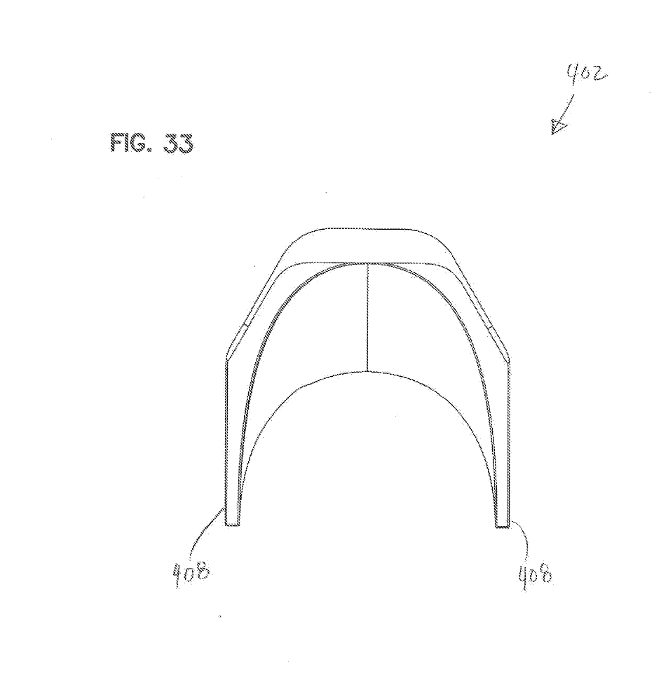

FIG. 27 is a front perspective view of the example therapeutic boot support 400. The heel support structure 404 is arranged and constructed to attach at the back of the heel float cradle 402 to form a cup like configuration thereon. The heel support structure 404 can be positioned at the bottom of the heel float cradle 402 to help protect the heel of the patient within the heel float therapeutic boot 300. The heel float cradle 402 is illustrated and described in more detail with reference to FIGS. 28-33.

Referring to FIGS. 28-33, features of the example heel float cradle 402 are illustrated.

The heel float cradle 402 includes a posterior flat surface 406 and side walls 408 that extend along the longitudinal sides of the lower leg. The posterior flat surface 406 stabilizes the heel float cradle 402 and lower leg against rotation when the patient rests in a supine position. The heel support structure 404 can be positioned on the posterior flat surface 406 and the side walls 408 of the heel float cradle 402.

The heel float cradle 402 provides a longitudinal channel 410 that receives the lower leg of a patient. In this example, the heel float cradle 402 has a U-shaped cross-section. In other embodiments, the heel float cradle 402 can have other cross-sectional shapes, such as, but not limited to, a semi-circular or rectangular shape.

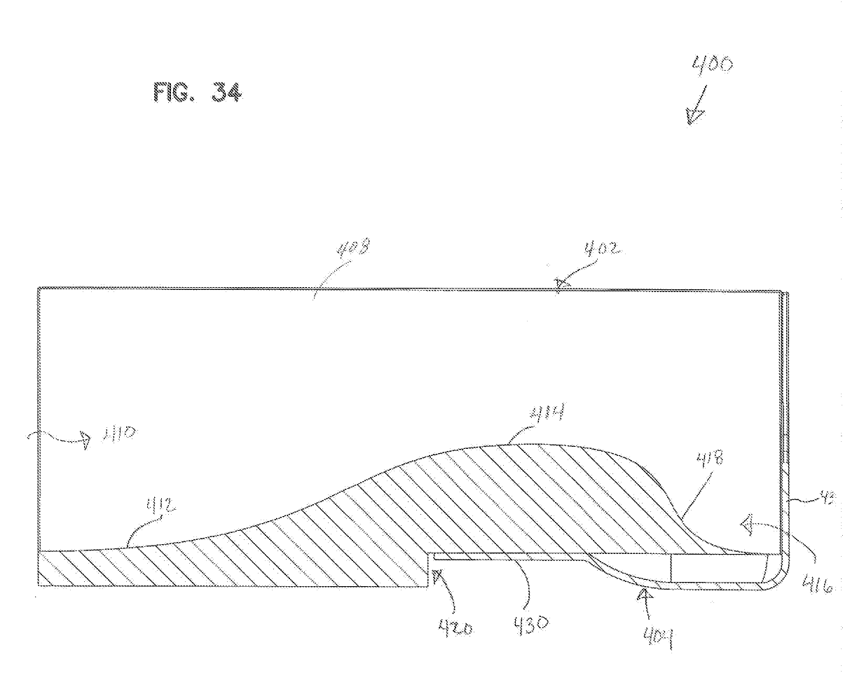

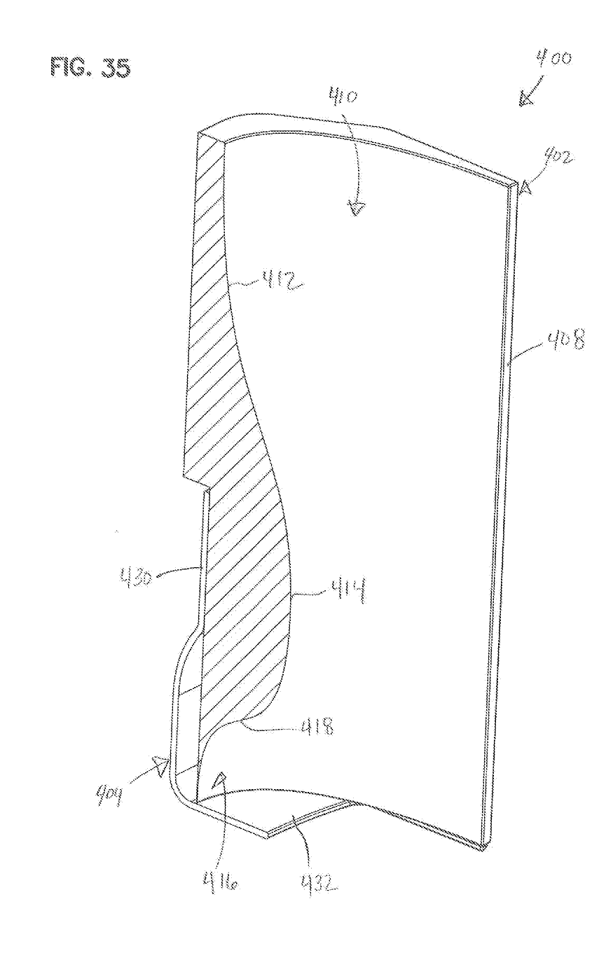

FIGS. 34-35 are cross-sectional views of the example therapeutic boot support 400.

As shown, the heel float cradle 402 includes an anterior surface 412 along the longitudinal channel 410, a raised surface portion 414, a recessed region 416, a peripheral edge 418 of the anterior surface 412 and a recess 420 formed into the posterior flat surface 406 of the heel float cradle 402. The recess 420 is adjacent the inverted U-shaped recessed region 416 adjacent to the peripheral edge 418 of the anterior surface 412 of the heel float cradle 402. Many of the possible aspects and features of the heel float cradle 402 are disclosed or are similar to the lower leg support portion 160 shown in FIGS. 6-7 and 11-12. Accordingly, the descriptions and features of these structures are not repeated here for the heel float cradle 402.

The recess 420 is constructed to receive the heel support structure 404 to help provide rigidity to the therapeutic boot support 400. As depicted, the heel support structure 404 can connect to the therapeutic boot support 400 and be used internally within the heel float therapeutic boot 300. In some examples, the recess 420 can be constructed to receive other components (e.g., foot support platform) in addition to the heel support structure 404. The heel float cradle 402 and the other component can be attached together at the recess 420 to create a hinge point. Many of the possible aspects and features for the heel support structure 404 are disclosed or are similar to the heel support structure 308 shown in FIGS. 20-23, for example. The heel support structure 404 is illustrated and described in detail with reference to FIGS. 36-42.

FIGS. 36-42 illustrate features of the example heel support structure 404.

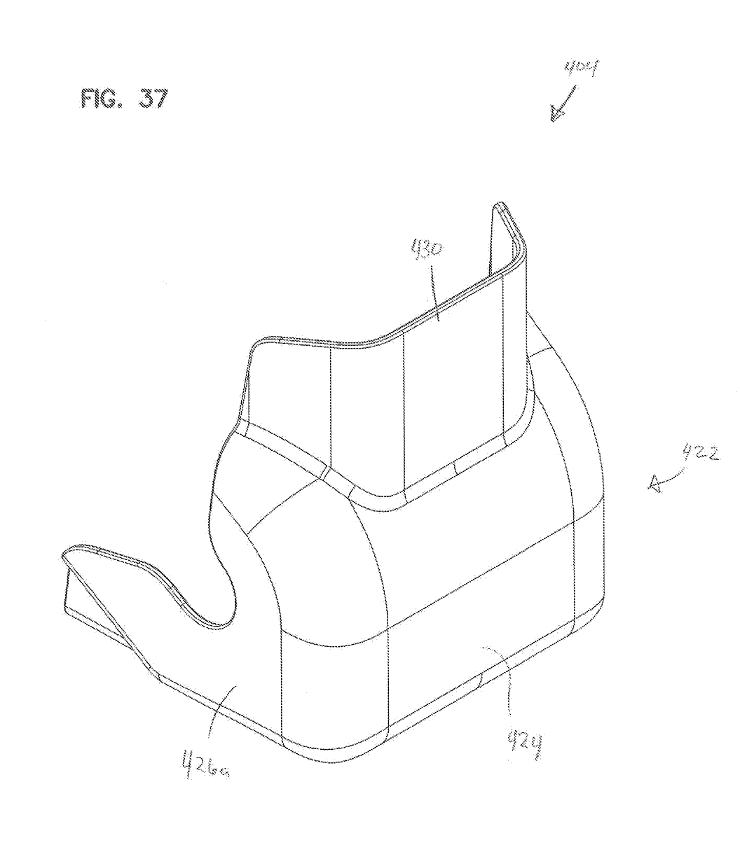

FIGS. 36-37 are perspective views of the example heel support structure 404. In this example, the heel support structure 404 includes a main body 422 having a posterior wall 424 and side walls 426 that extend outwardly from the posterior wall 424. In the depicted example, the side walls 426 include a side wall 426a and an oppositely disposed side wall 426b. The posterior wall 424 and the side walls 426a, 426b cooperatively define an interior region 428. The posterior wall 424 and the side walls 426 cooperatively further define a perimeter of the main body 422 of the heel support structure 404.

In some examples, the heel support structure 404 further includes a leg support panel 430 and a foot support panel 432 that each extend from the main body 422 of the heel support structure 404.

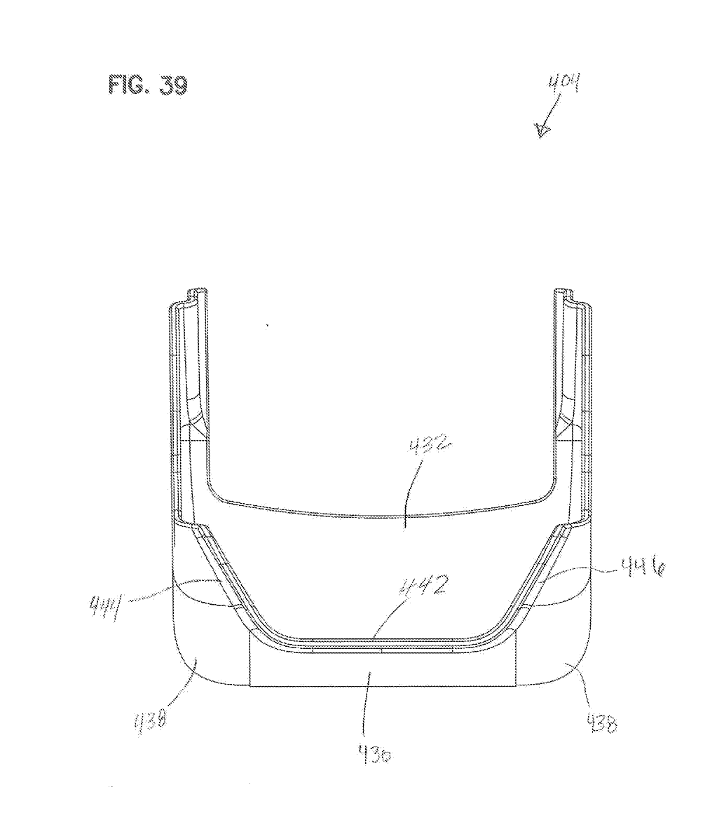

In one example, the leg support panel 430 can be arranged and configured to correspond with the recess 420 of the heel float cradle 402 to be positioned and fitted therein. In the depicted example, the side walls 426, the posterior wall 424, the leg support panel 430, and the foot support panel 432 are integral (e.g., one piece). The posterior wall 424, the side walls 426, the leg support panel 430, and the foot support panel 432 are illustrated and described in more detail with reference to FIGS. 39-43.

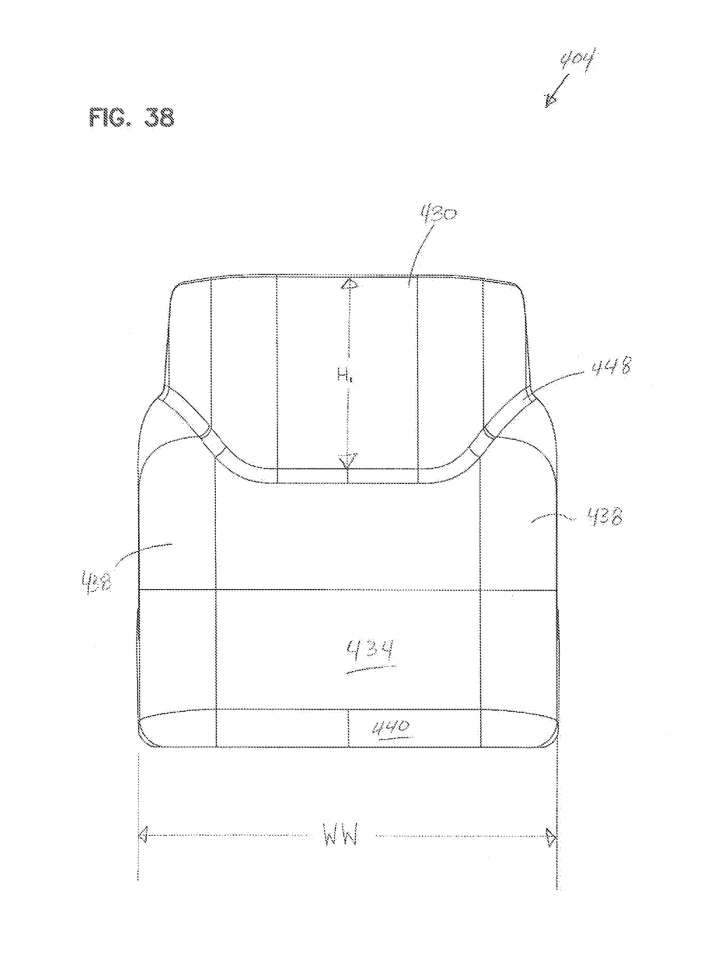

Referring to FIG. 38, a rear perspective view of the example heel support structure 404 is depicted. In this example, the posterior wall 424 has generally a flat exterior surface 434 and interior surface 436 (see FIG. 36). The posterior wall 424 can have rounded corners 438 that are integrally formed with the respective side walls 426a, 426b. The posterior wall 424 can further include a rounded bottom 440 integrally formed with the foot support panel 432 along the perimeter thereof. The size, geometry, and configuration of the rounded corners 438 and the rounded bottom 440 may vary in other embodiments. In the depicted example, the posterior wall 424 has a wall width WW measured between the side walls 426a, 426b. The geometry and size of the posterior wall 424 can vary with other embodiments. In the depicted example, the posterior wall 424 and the side walls 426a, 426b of the example heel support structure 404 has generally a squared shape. The general shape of the posterior wall 424 and the side walls 426a, 426b can vary with other embodiments. For example, the posterior wall 424 and the side walls 426a, 426b can have a semi-circular configuration.

Some embodiments include one or more apertures formed through the heel support structure 404 to provide passageways for air and moisture to flow through the heel support structure 404. As one example, the apertures can be formed in the exterior surface 434, support panel 432 (FIG. 39), and/or side walls 426a/426b, or elsewhere.

Referring to FIG. 39, a top perspective view of the example heel support structure 404 is depicted. In certain examples, the leg support panel 430 of the heel support structure 404 extends outwardly from the posterior wall 424 and the side walls 426. The leg support panel 430 can have a profile that generally corresponds with the posterior flat surface 406 and the side walls 408 of the heel float cradle 402. The leg support panel 430 can include a center portion 442, a left side portion 444, and a right side portion 446. In one example, the center portion 442 can be positioned adjacent to the posterior flat surface 406 of the heel float cradle 402. The left and right side portions 444, 446 can be connected to the side walls 408 respectively.

In one example, the left and right side portions 444, 446 extend from the center portion 442 of the leg support panel 430 and angle outwardly from each other. In one example, the left and right side portions 444, 446 can be angled at about 45 degrees relative to the center portion 442. In other embodiments, the angle and configuration of the left and right side portions 444, 446 can vary to be greater or smaller. The leg support panel 430 helps to secure the heel support structure 404 on the heel float cradle 402. In certain examples, the leg support panel 430 is generally parallel to the posterior wall 424. The leg support panel 430 extends up the lower leg of a patient having a height H₁ (see FIG. 38) at the center portion 442.

In some examples, the posterior wall 424 and the side walls 426 together define a groove 448 (see FIG. 38) between the center portion 442, the left side portion 444, and the right side portion 446 of the leg support panel 430.

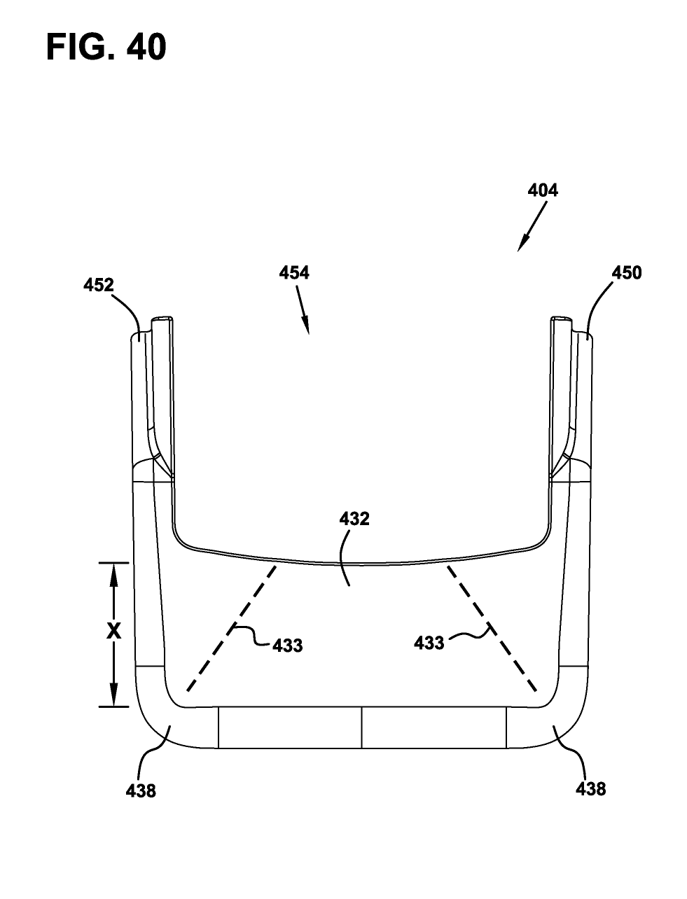

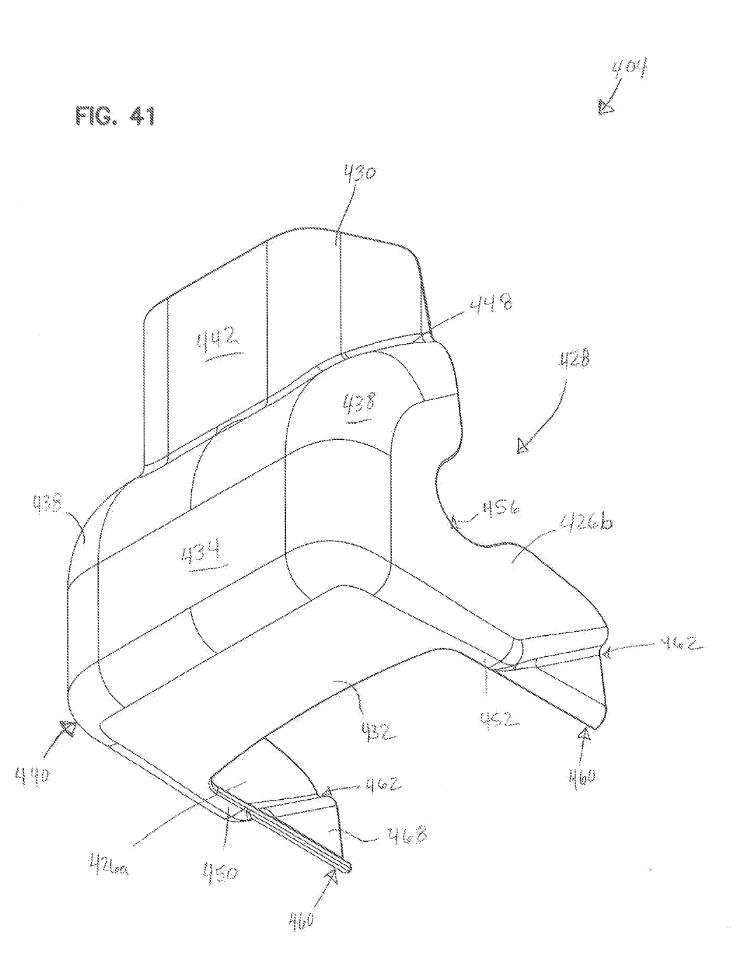

Referring now to FIGS. 40-41, a bottom plan view and a bottom perspective view of the example heel support structure 404 is depicted. In certain examples, the foot support panel 432 of the heel support structure 404 can be generally perpendicular to the posterior wall 424. The foot support panel 432 can be integrally formed or coupled with the rounded bottom of the posterior wall 424 and the side walls 426.

The foot support panel 432 can generally extend a distance X from the rounded bottom 440 along the foot of a patient. In some examples, the distance X of the foot support panel 432 can be made shorter to avoid extending along the foot of the patient. In one example, the foot support panel 432 extends just below the heel of the patient. In other examples, the foot support panel 432 extends to the heel of the patient. The foot support panel 432 is configured to eliminate any pressure points along the bottom of the foot of the patient.

The foot support panel 432 can include a left end 450 and an opposite right end 452 that together define an opening 454. In this example, the opening 454 helps to eliminate any pressure points that might induce skin ulcerations or abrasion on the foot of the patient. The opening 454 adjacent the foot support panel 432 helps to avoid the foot support panel 432 extending along the foot.

In some examples, the heel support structure 404 and the heel float cradle 402 can be covered with layers of material such as, non-wovens, fleece, fabric, and other materials described above for making the heel float therapeutic boot 300. For example, the therapeutic boot support 400 can be placed between any of the layers of materials in the lower leg support portion 302 or the foot support portion 304. The therapeutic boot support 400 may be covered on the exterior surface with a fabric or foam to prevent injury to other limbs while lying down in bed. The therapeutic boot support 400 may help to maintain or increase blood flow. The increase in blood circulation can minimize the potential of developing wounds or sores on the skin. The squared block shape of the heel support structure 404 helps to keep the foot of the patient upright and not roll over. It is to be understood that the size, geometry and configuration of the foot support panel 432 may vary with other examples.

In another possible embodiment, slits are formed in the support panel 432 along lines 433 (FIG. 40). The slits reduce the rigidity of the heel support structure 404 along the corners 438, allowing the heel support structure 404 to flex at the corners 438 as a living hinge. This provides added flexibility to the heel support structure 404 to accommodate patients of different sizes, and particularly patient's whose lower leg is larger than the average. More specifically, the slits allow the side walls to flex outward to increase the interior space within the heel support structure 404. In some embodiments the slits include one or more overlapping tabs that extend across the slits to cover the slits and prevent or reduce the chance of surrounding materials becoming lodged or pinched within the slit.

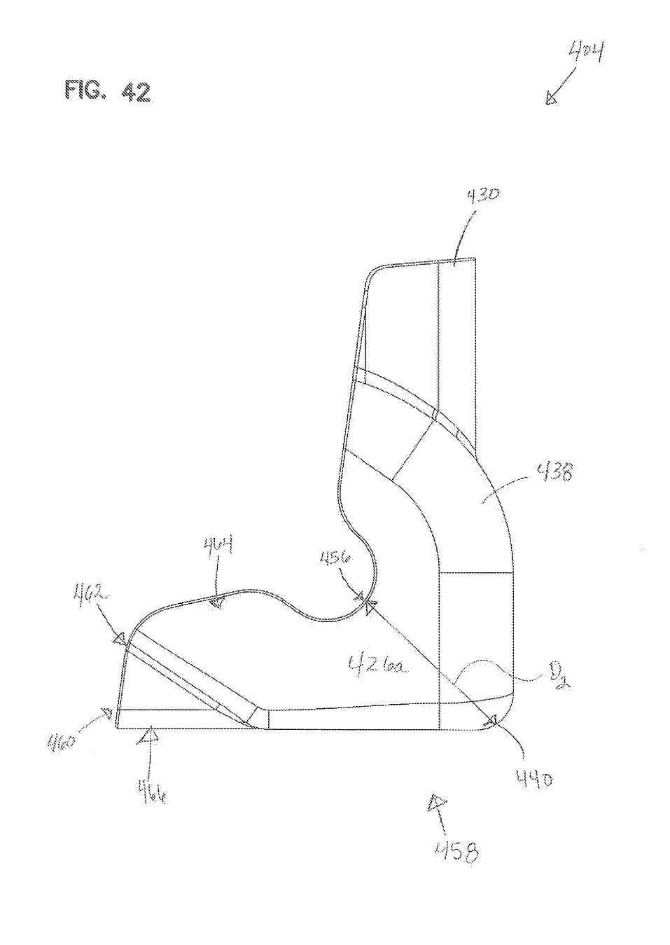

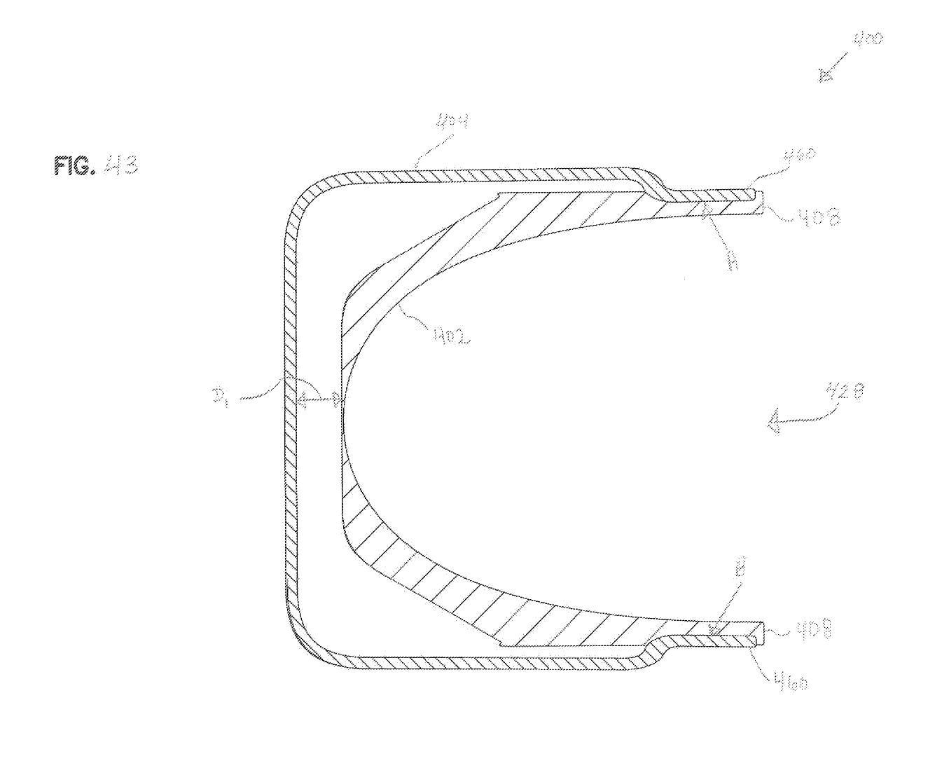

Referring now to FIG. 42, a side perspective view of the example heel support structure 404 is depicted, which includes side walls 426a, 426b. Although only the side wall 426a is visible in FIG. 42, the other side wall 426b is a mirror image in some embodiments.

In some examples, the side wall 426a is formed integrally or coupled with the rounded corners 438 and the rounded bottom 440 and extends therefrom to form a left side 458 of the heel support structure 404. The side wall 426a is constructed to help support and protect the lower leg and sides of the ankle while the foot is in an upright position or lying on its side. In this example, the side wall 426a provides a rigid support structure to function as a shield to help protect the patient from pressure sores.

In one example, the side wall 426a defines a notch 456 for providing access to the leg of the patient for routing hoses or tubing within the heel float therapeutic boot 300 whereby additional medical treatment can be provided such as a compression therapy device. In some examples, the hoses or tubing can be routed through the opening 454 adjacent the foot support panel 432. In other examples, the side wall 426a can include an integrally formed tab 460 that extends in a downward direction from a step 462 formed in the side wall 426a. The step 462 extends generally in a horizontal direction between a first position 464 of the side wall 426a and a second position 466 of the side wall 426a. The step 462 can be characterized as a transition point between the first and second positions 464, 466. The tab 460 can be integrally formed or coupled on both the side wall 426a and the side wall 426b. The tab 460 can be adapted to be secured to the side walls 408 of the heel float cradle 402.

In certain examples, the tab 460 can be secured with an attachment mechanism such as, but not limited to, adhesive. The adhesive can be applied onto a first surface 468 of the tab 460. Once the adhesive is applied on the first surface 468 of the tab 460, the heel support structure 404 can be positioned on the heel float cradle 402 such that the adhesive side of the tab 460 of the heel support structure 404 contacts the side walls 408 of the heel float cradle 402 to attach the heel support structure 404 thereon. In other examples, other attachment mechanisms can be used, such as, buttons, hooks, clips, clasps, bolts, straps, or combinations of these or other fastening mechanisms. In one example, the heel support structure 404 can be permanently attached to the heel float cradle 402.

Referring now to FIG. 43, a cross-sectional plan view of the therapeutic boot support 400 is depicted, which includes tabs 460. Adhesive, such as, double sided adhesive, can be applied to the tabs 460 for adhering the tabs 460 onto the side walls 408 of the heel support structure 404. The tabs 460 contact the heel float cradle 402 generally at points A, B. In certain examples, the attachment of the tabs 460 to the heel float cradle 402 at points A, B creates a suspension mechanism. The heel float cradle 402 spans the distance between points A, B, and is suspended within the interior region 428 of the heel support structure 404 such that there is no contact between the heel float cradle 402 and the heel support structure 404. The space between the heel float cradle 402 and the heel support structure 404 is defined as distance D₁. The suspension bridge mechanism provides and maintains this distance D₁, which helps suspend a foot of the patient while lying down. In other examples, the distance D₁ can be greater. In some examples, the distance D₁ may be smaller while maintaining no contact between the heel float cradle 402 and the heel support structure 404.

In one example, the heel support structure 404 can be a protection device and a built in suspension mechanism all in one structure or piece. The heel support structure 404 protects the heel and ankle within the heel float therapeutic boot 300. The heel support structure 404 can also suspend the foot, ankle, and heel of a patient using the tab 460 as a suspension mechanism. In other examples, the protection device and the suspension mechanism may be provided by a multiple piece heel support structure. In other examples, the heel support structure 404 may be attached to the heel float cradle 402 at a location other than at the tab 460. In other examples, the heel support structure 404 may be attached to the heel float cradle 402 at more than one location while the leg support panel 430 is positioned within the recess 420 of the heel float cradle 402.

In one example, the heel support structure 404 is a non-removable structure after being attached to the heel float cradle 402. In other embodiments, the heel support structure 404 is a removable structure. In certain embodiments, the heel support structure 404 is a multiple piece structure (e.g., two-piece) such that at least one of the pieces or all pieces can be removable.

The interior region 428 of the heel support structure 404 encloses the heel of the patient suspended above therein to help protect it from pressure points. In this example, the interior region 428 can have a depth distance D₂ (see FIG. 42) to receive the heel of a patient wearing the heel float therapeutic boot 300.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

What is claimed is:

1. A heel float therapeutic footwear apparatus comprising:
a lower leg support portion;
a foot support portion extending from the lower leg support portion;
a heel float cradle having a raised portion, the raised portion configured to elevate a heel of a patient when the patient is lying on the patient's back to reduce pressure on the patient's heel, the heel float cradle defining a recess; and
a portion of a heel support structure adapted to be received within the recess of the heel float cradle for protecting the heel and ankle of the patient, the heel support structure forming a suspension bridge thereon to suspend a foot of the patient.

2. The heel float therapeutic footwear apparatus according to claim 1, further comprising a foot support platform extending from the lower leg support cradle.

3. The heel float therapeutic footwear apparatus according to claim 2, further comprising a wing support structure attached at a first end portion to the foot support platform, and having a second end portion that is adjustably fastenable to a side of the lower leg support portion.

4. The heel float therapeutic footwear apparatus according to claim 2, wherein the foot support platform includes:
an extended flap portion being configured to align with a base of the foot support portion, the extended flap portion having a proximal end bonded to a recess formed into a posterior flat surface of the heel float cradle adjacent the recessed region;
flaps extending from opposite sides of the extended flap portion; and
foam being positioned on the extended flap portion;
wherein the heel float cradle and the foot support platform are connected at the recess.

5. The heel float therapeutic footwear apparatus according to claim 1, wherein the heel support structure includes:
a main body including a posterior wall and side walls that extend outwardly from the major wall, the side walls cooperating to define a perimeter of the main body.

6. The heel float therapeutic footwear apparatus according to claim 5, wherein the heel support structure further comprises an interior region being defined by the posterior wall and the side walls.

7. The heel float therapeutic footwear apparatus according to claim 5, wherein the heel support structure further comprises a tab attached to each of the side walls, wherein an attachment mechanism is used to secure the tab to side walls of the heel float cradle.

8. The heel float therapeutic footwear apparatus according to claim 1, wherein the heel support structure is constructed with a polymer material.

9. The heel float therapeutic footwear apparatus according to claim 5, wherein the major wall and the side walls are integrated to form one piece.

10. The heel float therapeutic footwear apparatus according to claim 7, wherein the attachment mechanism is an adhesive.

11. The heel float therapeutic footwear apparatus according to claim 5, wherein the main body has a generally square configuration.

12. The heel float therapeutic footwear apparatus according to claim 7, wherein the side walls each define a notch for routing tubing or hoses within the apparatus.

13. A heel float therapeutic footwear apparatus comprising:
a lower leg support portion configured to support a lower leg of a patient;
a foot support portion connected to and extending from the lower leg support portion;
a heel float cradle having a raised portion, the raised portion configured to elevate a heel of the patient when the patient is lying on the patient's back to reduce pressure on the patient's heel; and
a heel support structure configured to attach to the heel float cradle; the heel support structure being adapted to protect the heel of the patient, the heel support structure including:
a main body including a posterior wall and side walls that extend from the posterior wall, the side walls cooperating to define a perimeter of the main body;
an interior region being defined by the posterior wall and the side walls; and
a tab adapted to be attached to the side walls, wherein an attachment mechanism is used to secure the tab to the side walls of the heel float cradle forming a suspension bridge thereon to suspend a foot of the patient.

14. The heel float therapeutic footwear apparatus according to claim 13, wherein the posterior wall and the side walls are integrated to form one piece.

15. The heel float therapeutic footwear apparatus according to claim 13, wherein the attachment mechanism is an adhesive.

16. The heel float therapeutic footwear apparatus according to claim 13, wherein the main body has a generally square configuration.

17. The heel float therapeutic footwear apparatus according to claim 13, wherein the heel support structure is constructed with a polymer material.

18. A method of making a heel float therapeutic footwear apparatus, the method comprising:
forming a lower leg support portion and a foot support portion;
forming a heel float cradle within the lower leg support portion; the heel support cradle having a raised portion, the raised portion configured to elevate a heel of the patient when the patient is lying on the patient's back to reduce pressure on the patient's heel; and
permanently attaching a heel support structure on the heel support cradle, the heel support structure being adapted to suspend a foot and protect the heel of the patient.

19. The method of making a heel float therapeutic footwear apparatus according to claim 18, further comprising forming a wing support structure including a left support wing and a right support wing, each of the wings including a first end portion and a second end portion, wherein the second end portions are configured to be adjustably fastened to opposite sides of the lower leg support portion.

20. The method of making a heel float therapeutic footwear apparatus according to claim 19, further comprising permanently fastening first end portions of the left and right support wings to the foot support portion.

21. The method of making a heel float therapeutic footwear apparatus according to claim 18, wherein the heel support structure includes a main body including a posterior wall and side walls that extend from the posterior wall, the side walls cooperating to define a perimeter of the main body.

22. The method of making a heel float therapeutic footwear apparatus according to claim 21, wherein the heel support structure further includes an interior region being defined by the posterior wall and the side walls.

23. The method of making a heel float therapeutic footwear apparatus according to claim 22, wherein the heel support structure further includes a tab integral formed with the side walls, wherein the tab is adapted to be attached to a portion of the heel float cradle forming a suspension bridge thereon to suspend a foot of the patient.
